(19) United States
(12) Patent Application Publication
(10) Pub. No.: US 2024/0306646 A1
(43) Pub. Date: Sep. 19, 2024

(54) BROAD SPECTRUM PATHOGEN CONTROL IN PLANTS

(71) Applicants: APHEA. BIO NV, Gent (BE); FUNDACIÓN CENTRO DE EXCELENCIA EN INVESTIGACIÓN DE MEDICAMENTOS INNOVADORES EN ANDALUCÍA, MEDINA, Granada (ES)

(72) Inventors: Frederik ROELS, Sint-Denijs-Westrem (BE); Isabel VERCAUTEREN, Herzele (BE); Jens MAINTZ, Zwijnaarde (BE); Steven VANDENABEELE, Oudenaarde (BE); Emma LAGAE, Afsnee (BE); Thomas SIMON, Zwijnaarde (BE)

(21) Appl. No.: 18/571,132

(22) PCT Filed: Jun. 17, 2021
(86) PCT No.: PCT/EP2021/066372

§ 371 Date: Dec. 15, 2023

(51) Int. Cl. A01N 63/20; A01N 33/16; A01N 43/04; A01P 3/00; C07C 203/04

(52) U.S. Cl. CPC A01N 63/20; A01N 33/16; A01N 43/04; A01P 3/00; C07C 203/04

(57) ABSTRACT

The current invention relates to a purified bacterial strain for conferring a broad-spectrum pathogen control or resistance to a plant, wherein said strain comprises at least one 16S nucleotide sequence, said 16S nucleotide sequence shows at least 95% sequence identity to SEQ ID No 2 over the entire length of said sequence and comprises a nucleotide signature sequence according to SEQ ID No 1. The invention also relates to a bacterial population and to a formulation for conferring a broad-spectrum pathogen control or resistance to a plant, as well as to the use of such purified bacterial strain, bacterial population or formulation to control a pathogen in a plant. In addition, the invention relates to a compound having the formula C21H23NO4. The invention also relates to a method for conferring a broad-spectrum pathogen control or resistance in a plant, and to the use of Takanawaene and/or C21H23NO4 compounds for controlling a pathogen in a plant.

### FIELD OF THE INVENTION

The present invention relates to the field of pathogen control and resistance in plants using bacteria. The present invention discloses both means and methods for controlling these pathogens.

### BACKGROUND

There is an increasing demand for agricultural products, in order to be able to feed the growing human population and to feed cattle. In addition, such growing demand puts a further strain on the environment due to the conventional practices used in agriculture. The most serious problem encountered in the cultivation of crops is the loss of plant material and damage caused by harmful and often destructive plant pathogens.

Various methods have been developed so far to control plant diseases. Among them, the most commonly used and the most developed method is controlling plant diseases by using chemical pesticides. Chemical pesticides are convenient to use and can have immediate effects to protect plants from pests, however over recent years several active substances have been subject to regulatory action. Several active substances have been banned for use and products have been withdrawn, for other active substances the maximum rates have been lowered. In recent years, the misuse of chemical pesticides has created social problems such as intoxications and deaths caused by acute toxicity; contamination of food due to active substances exceeding the maximum residue levels in agricultural products; incidences with end users being exposed to chemical pesticides, and damage to the environment due to spillages and drift. Furthermore, pests may develop resistance to chemical pesticides, forcing the development of new types of pesticides.

A promising practice is the use of microorganisms for antimicrobial activities. WO 2017 019 633 for instance describes the use of isolated microorganisms, microbial consortia and agricultural compositions comprising the same for imparting beneficial properties, like improving resistance to pathogens, in plants. U.S. Pat. No. 7,037,879 discloses the use of endophytic bacteria for conferring pest resistance in plants. Plants infected with microorganisms have the above-mentioned resistance conferred by the endophytes. Chemical pesticides are often no longer required for cultivating these plants, which indicates that prevention of pests and diseases can be possible by treating plants with microorganisms.

However there is a further demand for other bio-based pathogen control methods and means, preferably broad-spectrum methods that are not harmful for the environment and the consumer. The current invention aims to provide a solution for the latter.

### SUMMARY OF THE INVENTION

The present invention and embodiments thereof serve to provide a solution to one or more of the above-mentioned disadvantages. To this end, the present invention relates to a purified bacterial strain for conferring a broad-spectrum pathogen control or resistance to a plant according to claim 1. Preferred embodiments of the purified bacterial strain are shown in any of the claims 2 to 9.

In a second aspect, the present invention relates to a bacterial population according to claim 10. A third aspect of the present invention relates to a formulation according to claim 11-13. In a further aspect, the present invention relates to a plant part according to claim 14. In a following aspect, the present invention relates to a use according to claim 15, according to claims 17-18 and according to claim 23. In addition, an aspect of the invention relates to method according to claim 16.

In a final aspect, the present invention relates to a compound having the formula C21H23NO4 according to claims 19-22.

### DESCRIPTION OF FIGURES

The following description of the figures of specific embodiments of the invention is merely exemplary in nature and is not intended to limit the present teachings, their application or uses.

FIG. 1 shows a graphical representation of biocontrol in Lactuca sativa. The impact of treatment with B/00227 on total yield of Lactuca sativa has been compared to the control treatment (Mock, consisting of the formulation only, dotted red line, normalized) (FIG. 1A). Yield is defined as fresh weight of the disease free part of the lettuce heads treated versus the mock treated. FIG. 1B shows the inhibitory effects on disease symptoms by treatment with either a chemical control (Signum+Ortiva) or with B/00227 compared to the mock treatment (formulation only, dotted, normalized).

FIG. 2 shows a graphical representation of the reduction of Fusarium graminearum (Fg) BRFM1977 (FIG. 2A) and Fusarium culmorum BRFM1944 (FIG. 2B) in infected Triticum aestivum spikes caused by treatment with B/00227 and B/00228 cells. A) Upper graph: The Y-axis visualizes the severity of the disease (0.0 is no disease; 1.0 is 100% Fusarium diseased wheat spikes), and the X-axis visualizes the five different treatments of the wheat plant samples. Lower graph: The Y-axis visualizes the difference in disease compared to the untreated/mock control. BFRM1977 means that the wheat plant spikes have been infected only with Fusarium graminearum BFRM1977 (Fg; on 55 replicated spikes); B/00227 and B/00228 means that the wheat plant spikes have been treated with the bacterial strains as given Deposit ID B/00227 (on 54 replicated spikes) and ID B/00228 (on 55 replicated spikes) respectively after Fg infection; Prosaro means that the Fg infected wheat plants have been treated with a chemical called Prosaro as positive control after Fg infection (on 10 replicated spikes), in accordance to the guidelines of the manufacturer. Mock means that the wheat plant has not been infected with Fg but has been treated with a mock sample (formulation only, on 18 replicated spikes). The figures visualize the disease assessment of an experiment at +20 days post infection (dpi). B) The Y-axis visualizes the severity of the disease, the X-axis visualizes three different treatments of the wheat plant samples. BFRM1944 means that the wheat plant spikes have been infected only with Fusarium culmorum (Fc; on 29 replicated spikes); B/00227 means that the wheat plant spikes have been treated with the bacterial strains as given Deposit ID B/00227 (on 12 replicated spikes) after Fc infection; Mock means that the wheat plant has not been infected with Fg but has been treated with a mock sample (formulation only, on 10 replicated spikes). The figures visualize the disease assessment of an experiment at +17 days post infection (dpi).

FIG. 3 shows a graphical representation of the reduction of Fusarium graminearum BRFM1977 in infected Triticum aestivum spikes spikes caused by treatment with B/00225 cells. Upper graph: The Y-axis visualizes the severity of the disease (0.0 is no disease; 1.0 is 100% Fusarium diseased wheat spikes), and the X-axis visualizes the 4 different treatments of the wheat plant samples. Lower graph: The Y-axis visualizes the difference to the untreated/mock control. BFRM1977 (infected only with Fusarium graminearum; Fg) was replicated on 54 spikes; B/00225 was replicated on 14 spikes after Fg infection; Prosaro was replicated on 10 replicated spikes after Fg infection. Mock (formulation only) was replicated on 10 spikes. These figure visualizes the disease assessment of an experiment at +21 days post infection (dpi).

FIG. 4 shows a graphical representation of the reduction of Fusarium graminearum BRFM1977 (FIG. 4A) and Fusarium culmorum BRFM1944 (FIG. 4B) in infected Triticum aestivum spikes caused by treatment with B/00226 cells. A) Upper graph: The Y-axis visualizes the severity of the disease (0.0 is no disease; 1.0 is 100% Fusarium diseased wheat spikes), and the X-axis visualizes the five different treatments of the wheat plant samples. Lower graph: The Y-axis visualizes the difference to the untreated/mock control. BFRM1977 (infected only with Fusarium graminearum; Fg) was replicated on 40 spikes; B/00226 was replicated on 15 spikes after Fg infection; Prosaro means that the Fg infected wheat plants have been treated with a chemical called Prosaro as positive control after Fg infection (on 12 replicated spikes). Mock (formulation only) was replicated on 8 spikes. This figure visualizes the disease assessment of an experiment at +22 days post infection (dpi). B) The Y-axis visualizes the severity of the disease, the X-axis visualizes three different treatments of the wheat plant samples. BFRM1944 (infected only with Fusarium culmorum; Fc) was replicated on 19 replicated spikes; B/00226 was replicated on 18 spikes after Fc infection; Mock means that the wheat plant has not been infected with Fg but has been treated with a mock sample (formulation only, on 10 replicated spikes). The figures visualize the disease assessment of an experiment at +7 days post infection (dpi).

FIG. 5 shows two graphical representations of the reduction of Fusarium graminearum BRFM1995 in infected Triticum aestivum spikes caused by treatment with B/00225, B/00226 and B/00227 spores. The Y-axis visualizes the severity of the disease, and the X-axis visualizes the different treatments of the wheat plant samples being Fusarium graminearum BRFM1955 only, B/00225 (FIG. 5B), B/00226 (FIG. 5B) and B/00227 (FIG. 5A) and Mock. This figure visualizes the biocontrol activity of B/00225 (on 10 replicated spikes), B/00226 (on 12 replicated spikes), B/00227 (on 16 replicated spikes) against F. graminearum at +19 dpi (FIG. 5A) and 12 dpi (FIG. 5B) of infected T. aestivum spikes. BFRM1955 means that the wheat plant spikes have been infected only with Fusarium graminearum (Fg; on 37 and 39 replicated spikes in Figure A and Figure B respectively). Mock means that the wheat plant has not been infected with Fg but has been treated with a mock sample (formulation only, on 9 and 5 replicated spikes in Figure A and Figure B respectively).

FIG. 6 shows the 16 nucleotides long signature sequence (SEQ ID No1) that is conserved in the 16S sequence of the deposited strains B/00225, B/00226, B/00227 B/00228, B/00231. This sequence is unique only for the Kitasatospora strains showing biocontrol activity against phytopathogens and therefore defines a specific group within the Kitasatospora genus that can be applied as biocontrol agent.

FIG. 7A visualizes the typical UV spectrum of the metabolite of B/000227 showing suppressive activity against phytopathogens where the shape and structure of the peak undeniably indicates the presence of 5 conjugated double bonds. FIG. 7B shows mass spectrometry spectrum of the active metabolite identified in B/00225, B/00226 and B/00227 showing suppressive activity against phytopathogens. The active metabolite is identified as Takanawaene C.

FIG. 8 shows the chemical structure of a Takanawaene (8A) and Takanawaene C (8B) compound.

FIG. 9 shows a graphical representation of the reduction of the fungal disease Fusarium head blight severity caused by Fusarium sp. in Triticum sp. in field conditions. The Y-axis visualizes the severity of the disease (score 0 to 100) averaged from 4 replicated plots of 24 square meters, and the X-axis visualizes the different treatments of the wheat plant samples being (1) untreated, (2) Prosaro (chemical positive control), (3) B/00225 and (4) B/00227. Disease severity was scored 4 weeks after application of B/00225, B/00227 and Prosaro. B/00225 and B/00227 was applied as a sprayable at the flowering stage of the wheat plants. Application was performed at 2 different timepoints (first at BBCH 61 (10% anthesis), equivalent to the beginning of flowering and at BBCH65, equivalent to 50% anthesis.) to cover small heterogeneities in flowering amongst the different wheat plants in a plot. All treatments were replicated 4 times in the field and disease scorings averaged of all replicated plots. The effects size disease reduction was up to 89% of the chemical control Prosaro.

### DEFINITIONS

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, 6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

An “endophyte” is an organism capable of living on a plant element (e.g. rhizoplane or phyllosphere) or within a plant element (e.g. endosphere) or on a surface in close physical proximity with a plant element (e.g. the rhizosphere or on a seed). Endophytes can occupy the intracellular or extracellular spaces of plant tissue, including but not limited to leaves, stems, flowers, fruits, seeds, or roots. An endophyte can be, for example, a bacterial or fungal organism, and can confer a beneficial property to the host plant such as an increase in yield, biomass, resistance, or fitness. An endophyte can be a fungus or a bacterium. As used herein, the term “microbe” or “microbial strain” is sometimes used to describe an endophyte.

As used herein, the term “microorganism”, “microbial strain” or “microbe” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte, for example a bacterial or fungal endophyte, which is capable of living within a plant. In some embodiments, a microbe or microorganism encompasses individual cells (e.g., unicellular microorganisms) or more than one cell (e.g., multi-cellular microorganism).

As used herein, the term “bacterium”, “bacteria”, or “bacterial” refers in general to any prokaryotic organism, and may reference an organism from Kingdom Eubacteria (Bacteria). In some cases, bacterial genera have been reassigned due to various reasons (such as, but not limited to, the evolving field of whole genome sequencing), and it is understood that such nomenclature reassignments are within the scope of any claimed genus.

The term “pathogen” or “plant pathogen” are known to be associated with the plant, its associated results, refers to organisms that cause harmful effects to the health and vigor of plants. Plant pathogens include fungi, bacteria, viruses, insects, nematodes, and the like.

The term “16S nucleotide sequence” or “16S” refers to the DNA sequence of the 16S ribosomal RNA (rRNA) sequence of a bacterium. 16S rRNA gene sequencing is a well-established method for studying phylogeny and taxonomy of bacteria. A full length 16S nucleic acid sequence counts for approximately 1500 nucleotides in length.

“Biomass” means the total mass or weight (fresh or dry), at a given time, of a plant tissue, plant tissues, an entire plant, or population of plants. Biomass is usually given as weight per unit area. The term may also refer to all the plants or species in the community (community biomass).

The term “purified” is intended to specifically reference an organism, cell, tissue, polynucleotide, or polypeptide that is removed from its original source. The term “purified” does not necessarily reflect the extent to which the microbe has been purified.

As used herein, a “purified bacterial strain” is a bacterial strain that has been removed from its natural milieu. The term refers to substantially no other bacterial strain than the desired one, and is therefore substantially free of other contaminants, which can include microbial contaminants. Further, as used herein, “purified bacterial strain” is intended to mean the bacterial strain separated from materials with which it is normally found in nature.

A “plant” includes any plant, particularly a plant of agronomic importance, within which or onto which a microbe, is heterologously disposed. As used herein, an endophyte is said to colonize a plant, plant element, root or seed, when it can exist as an endophyte in relationship with a plant or plant element during at least part of either the plant's or the microbe's life cycle. In some embodiments, an endophyte is said to “colonize” a plant or plant element when it can be stably detected within the plant or plant element over a period time, such as one or more days, weeks, months or years. Some of the compositions and methods described herein involve a plurality of microbes in an amount effective to colonize a plant and its offspring.

The terms “identity” or “identical” in the context of nucleotide sequences refer to the nucleotides in the two sequences that are the same when aligned for maximum correspondence. There are different algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm. For example, a local alignment may be generated using an implementation of the Smith-Waterman algorithm. A gap is a region of an alignment wherein a sequence does not align to a position in the other sequence of the alignment. In global alignments, terminal gaps are discarded before identity is calculated. For both local and global alignments, internal gaps are counted as differences. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment.

The term “reference plant” or “reference” is a comparative term, and reference plants that are genetically identical, but may differ in treatment. In one example, two genetically identical plant embryos (e.g. from wheat, or any other plant) may be separated into two different groups, one receiving a treatment (such as transformation with a heterologous polynucleotide, to create a genetically modified plant) and one control, e.g., reference, that does not receive such treatment. Any phenotypic differences, e.g. an improved resistance to a pathogen, between the two groups may thus be attributed solely to the treatment and not to any inherency of the plant's genetic makeup. In another example, two genetically identical wheat seeds may be treated with a formulation, one that introduces a microorganisms population and one that does not. Any phenotypic differences, e.g. an improved resistance to a pathogen, between the plants derived from (e.g., grown from or obtained from) those seeds may be attributed to the microbial treatment.

A “plant part” is intended to generically reference either a whole plant or a plant component, including but not limited to plant tissues, parts, and cell types. A plant part is preferably one of the following: whole plant, seedling, meristematic tissue, ground tissue, vascular tissue, dermal tissue, seed, leaf, root, shoot, stem, flower, ear, spike, spikelet, fruit, stolon, bulb, tuber, corm, keikis, bud. As used herein, a “plant part” refers to any part of the plant, and can include distinct tissues and/or organs, and may be used interchangeably with the term “tissue” throughout. In addition, a “plant element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keikis, or bud.

The term “agricultural plants” includes plants that are cultivated by humans for but not limited to food, feed, fiber, fuel, and/or industrial purposes. In some embodiments, plants (including seeds and other plant elements) treated in accordance with the present invention are monocots or dicots.

The term “application of microorganism” or “microorganism administration” means application of the strain and/or strains and or strain's extract in any form. These forms include but not limited to application of live cells, dead cells, broth, spores, extract; alone and/or in combination with other substances via spraying, coating, covering, contacting and immersion.

The term “spraying” means dispersing a substance. Spraying is, in agriculture, a standard method of applying, for example, pest-control chemicals and other compounds. In spraying, the compounds to be applied are preferably dissolved or suspended in a carrier, such as in a water- or, less commonly, in an oil-based carrier.

Said compounds may comprise, but are not limited to, microorganisms. The sprayed substance can contain microorganism (in any form) alone or in combination with other materials, and may be applied to a surface.

The term “sprayable” form refers to a substance that can be sprayed, for example on a surface. Sprayable forms of microorganisms include but not limited to live cells, spray-dried cells, spray-dried spores, freeze-dried cells, spores and freeze-dried spores.

The term “surface” refers to the outside part or uppermost layer (or a combination of different layers), of ‘something’. In the context of the present application, said ‘something’ may include but is not limited to growth medium (such as soil), plant, plant part, seeds, harvested crop, harvested seed crop, stored crops or crop parts.

An “active formulation” refers to a mixture of compounds that facilitates, for example, the stability, storage, and/or application of the bacterial strain.

As used herein an “agriculturally compatible carrier” refers to any material, that can be added to a plant, plant part or substrate comprising or hosting the plant or plant part without causing or having an adverse effect.

As used herein, a “colony-forming unit” or “CFU” is used as a measure of viable microorganisms in a sample. A CFU is an individual viable cell capable of forming on a solid medium a visible colony whose individual cells are derived by cell division from one parental cell.

The term “supernatant” refers to the liquid broth remaining when cells grown in said broth are removed by centrifugation, filtration, sedimentation or other means well known in the art.

The term “extract” refers to various forms of microbial products. Said microbial products are obtained by removing the cell walls and/or cell membranes of the bacterial endophytes, a process known as lysis. Thereby obtaining one or more endogenous products of the bacterial endophytes in culture.

The term “Polyene” refers to a poly-unsaturated organic compounds that contains at least three alternating double and single carbon-carbon bonds. These carbon-carbon double bonds interact in a process known as conjugation resulting in some unusual optical properties.

The term “Polyene macrolide” refers to a type of polyenes, and is a specific class of compounds that consists of a large substituted ring structure (macrolactone=macrocyclic lactone) with a series of double bonds (hence polyenes), such as trienes, tetraenes and pentaenes etc. and a series of hydroxyl groups in positions opposite to the double bonds. Other common substituents are a carboxyl group and an aminosugar-mycosamine. The structure has a specific UV spectrum which is dependent on the total amount of double bonds in the ring, generally between 3 and 8. The entire ring structure is synthesized by enzymes called Type 1 Polyketide Synthases (T1PKS). Certain polyene macrolides belong to a class of antimicrobial compounds that target fungi.

The term “Type 1 Polyketide Synthase (T1PKS)” refers to a gene. Polyketide synthases (PKSs) are a family of multi-domain enzymes or enzyme complexes that produce polyketides, a large class of secondary metabolites, in bacteria, fungi, plants, and a few animal lineages. Type I polyketide synthases are large, highly modular proteins. Each type I polyketide-synthase module consists of several domains with defined functions, separated by short spacer regions. The polyketide synthases (PKS) synthesize and assemble the macrolactone rings of macrolide polyenes enzymes by performing decarboxylative condensation of small (di)carboxylic acids, thus building a polyketide chain.

As used herein, a microbe, plant, or plant part is “modified” when it comprises an artificially introduced genetic or epigenetic “modification”. In some embodiments, the modification is introduced by a genome engineering technology. In some embodiments, the modification is introduced by a targeted nuclease. In some embodiments, targeted nucleases include, but are not limited to, transcription activator-like effector nuclease (TALEN), zinc finger nuclease (ZNF), clustered regulatory interspaced short palindromic repeats (CRISPR), CRISPR/Cas9, CRISPR/CPFL and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified microbe, plant, or plant element comprises a transgene.

Microorganisms can also be “heterologously disposed” on a given plant tissue. This means that the microorganism is placed upon a plant tissue where it is not naturally found upon. As such, a microorganism is deemed heterologously disposed, when applied on a plant that does not naturally have the microorganism present or does not naturally have the microorganism present in the number that is being applied.

As used herein “inhibiting” or “suppressing” and like terms should not be construed to require complete inhibition or suppression, although this may be desired in some embodiments.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of the primer. A pair of bi-directional primers consists of one forward and one reverse primer as commonly used in the art of DNA amplification such as in PCR amplification.

### DETAILED DESCRIPTION OF THE INVENTION

The present invention utilizes microorganisms to impart beneficial properties in plants, in particular but not limited to agricultural plants. The invention therefore offers an environmentally sustainable solution, which is not reliant or solely reliant upon utilization of synthetic herbicides and pesticides.

In a first aspect, the invention relates to a purified bacterial strain for conferring a broad-spectrum pathogen control or resistance to a plant, wherein said strain comprises at least one 16S nucleotide sequence, said 16S nucleotide sequence shows at least 95% sequence identity to SEQ ID No 2 over the entire length of said sequence, and wherein said 16S nucleotide sequence comprises a nucleotide signature sequence according to SEQ ID No 1.

It was shown that bacterial strains having at least one 16S nucleotide sequence which shows at least 95% sequence identity to SEQ ID No 2 over the entire length of SEQ ID No 2 and which comprises a nucleotide sequence according to SEQ ID No 1 as signature sequence exhibit an unexpected activity as broad-spectrum pathogen control agents.

In an preferred embodiment, the 16S nucleotide sequence exhibits at least 95% sequence identity to SEQ ID No 2. Preferably, the 16S nucleotide sequence exhibits at least 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, more preferably 100.0% sequence identity to SEQ ID No 2. Most preferably, the 16S nucleotide sequence is thus identical to SEQ ID No 2.

As indicated, the at least one 16S nucleotide sequence of the bacterial strains of the invention comprises a nucleotide signature sequence according to SEQ ID No 1. It was shown that species having this nucleotide signature sequence in their 16S nucleotide sequence exhibit an unexpected activity as broad-spectrum pathogen control agents. The inventors also found that strains which lack the full nucleotide signature sequence as given in SEQ ID No 1 in their 16S nucleotide sequence do not confer pathogen control or resistance to plants.

Without wishing to be bound to a theory, it seems that this specific sequence demarks a group of strains with an exceptional broad-spectrum pathogen control activity of said strains.

In an embodiment, said broad-spectrum pathogen control or resistance to the plant resistance is effective both in vitro and as in vivo.

The present invention relates to isolated and biologically pure microorganisms that have application, inter alia, in agriculture or gardening. The microbial strain is preferably isolated and purified from its natural environment. The microbial strain is an identifiable strain of a taxonomic species of bacteria.

The 16S nucleotide sequence analysis revealed that the microorganisms of current invention are related to the genus Kitasatospora however, the strains disclosed in this invention define a novel, separate subgroup within the Kitasatospora (‘Kitasatospora-like’) genus. Analysis of the 16S nucleotide sequence is widely used for phylogenetic studies as it is highly conserved between different species of bacteria. The genus Kitasatospora currently includes aerobic, Gram-positive, non-motile, chemo-organotrophic actinomycetes. Different species of Kitasatospora are isolated from soil samples and have shown among others an inhibitory activity against pathogens.

In an embodiment, the invention relates to a purified bacterial strain related to genus Kitasatospora for conferring a broad-spectrum pathogen control or resistance to a plant, wherein said strain comprises at least one 16S nucleotide sequence having at least 95% sequence identity to SEQ ID No 2 (over its full length), wherein said 16S nucleotide sequence comprises a nucleotide signature sequence according to SEQ ID No 1. This 16 nucleotide long signature sequence is preferably fully conserved in the microorganisms of the current invention, the presence or absence of it corresponds to the Kitasatospora-like strains showing biocontrol activity against phytopathogens or not respectively.

In a preferred embodiment, the 16S nucleotide sequence identity is determined over a region of alignment considering the full length 16S nucleotide sequence of SEQ ID No 2.

Next to its high conserved regions the 16S nucleotide sequence contains also hypervariable regions that provide species-specific signature sequences useful for identification of bacteria. A total of nine hypervariable regions (V1-V9) are considered, ranging from about 30 to 100 base pairs long.

In an embodiment said nucleotide signature sequence is located on the V6 region of said 16S nucleotide sequence. It was shown that all the identified strains as described herein comprise said signature sequence in the V6 region of the 16S confer broad-spectrum pathogen control or resistance to plants.

Although V6 regions contain the greatest intraspecies diversity, SEQ ID No 1 is reported by current invention clustering Kitasatospora-like strains in a group, wherein said Kitasatospora-like strains confer broad-spectrum pathogen control or resistance to plants.

In a more preferred embodiment of the current invention, said signature sequence is located in a nucleotide region which exhibits at least 95% sequence identity to SEQ ID No 4.

Preferably the nucleotide region of said bacterial strains has sequence identity of at least 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%. More preferably, the nucleotide region of said bacterial strains has 100.0% sequence identity with SEQ ID No 4.

Bacterial strains having sequence similarity with the nucleotide region as specified above or having said nucleotide region as given in SEQ ID No 4 confer a broad-spectrum pathogen control or resistance to plants.

The defined region given by SEQ ID No 4 is (part of) the V6 region of a 16S nucleotide sequence of the deposited strains B/00227, B/00225, B/00228, B/00226 and B/00231. Said defined region comprises the signature sequence as given by SEQ ID No 1. Said defined region is about 205 base pairs long and is defined between a set of primers, being 907F and 1100R. The set of primers is described in more detail below.

In another preferred embodiment, said V6 region comprises a sequence which has at least 95% sequence identity to SEQ ID No 4. Preferably said sequence has at least 95% sequence identity up to 100% sequence identity to SEQ ID No 4.

The purified bacterial strains of current invention were identified via whole genome sequencing. Each strain represents a pure colony isolate selected from an agar plate.

In another or further embodiment, the purified bacterial strains of the current invention express a polyene having antifungal activity.

Said polyene may be any type of polyene. Types of polyenes are known in the art.

In a preferred embodiment, said polyene is a polyene macrolide.

In another or further embodiment, said polyene is Takanawaene. Takanawaene is a polyene macrolide compound. In an embodiment, said Takananweane may be chosen from Takanawaene A, Takanawaene B or Takanawaene C, preferably said polyene is Takanawaene C.

As indicated before, polyene macrolides are a specific class of compounds that consists of a large substituted ring structure (macrolactone=macrocyclic lactone) with a series of double bonds and a series of hydroxyl group in positions opposite to the double bonds. The entire ring structure is synthesized by enzymes called Type 1 Polyketide Synthases (T1PKS). Certain polyene macrolides belong to a class of antimicrobial compounds that target fungi.

In another or further embodiment, the purified bacterial strains of the present invention have in their genome a gene sequence having at least 75%, and more preferably at least 77%, most preferably at least 80% of sequence identity with SEQ ID No 3. In a further preferred embodiment, said gene sequence is involved in the production pathway of said polyene.

In another or further embodiment, said polyene is Takanawaene. Takanawaene is a polyene macrolide compound. In an embodiment, said Takanawaene may be chosen from Takanawaene A, Takanawaene B or Takanawaene C, preferably said polyene is Takanawaene C.

In another or further embodiment, the purified bacterial strains of the current invention produce/express a compound having the formula C21H23NO4. Said compound was previously unknown, and is not disclosed in the Natural Products Databases. In addition, said compound has been shown to have antifungal and/or antibacterial activity against one or more pathogens, preferably plant pathogens.

In a particular preferred embodiment, the present invention relates to a purified bacterial strain for conferring a broad-spectrum pathogen control or resistance to a plant, wherein said strain comprises at least one 16S nucleotide sequence exhibiting at least 95% sequence identity to SEQ ID No 2, said 16S nucleotide sequence preferably is identical to SEQ ID No 2; and comprises a signature sequence according to SEQ ID No 1 and wherein said strain expresses a polyene, preferably Takanawaene A, B, or C, more preferably Takanawaene C, having antifungal activity.

Preferably the purified bacterial strains of the present invention have in their genome a gene sequence having at least 75%, more preferably at least 77%, most preferably 80% of sequence identity with SEQ ID No 3. In a further preferred embodiment, said gene sequence is involved in the production pathway of said polyene. In another or further embodiment, said purified bacterial strain expresses a compound having the formula C21H23NO4.

Examples of species according to the current invention have been deposited in the Polish Collection of Microorganisms, under the terms of the Budapest Treaty with deposit IDs B/00227, B/00225, B/00228, B/00226, or B/00231.

In an embodiment the purified bacterial strain is selected from the group consisting of Deposit ID B/00227, B/00225, B/00228, B/00226, or B/00231 or any mixtures thereof obtainable from the Polish Collection of Microorganisms, under the terms of the Budapest Treaty.

In an embodiment, the purified bacterial strain is a mutant of a strain deposited under B/00227, B/00225, B/00228, B/00226, or B/00231. In a preferred embodiment, said mutant is still capable of conferring a broad-spectrum pathogen control or resistance to a plant, preferably in a manner similar to said deposited strains.

In an embodiment, said purified bacterial strain has at least 98% genomic sequence identity with B/00227, B/00225, B/00228, B/00226, or B/00231. In a preferred embodiment, said strain is still capable of conferring a broad-spectrum pathogen control or resistance to a plant, preferably in a manner similar to said deposited strains.

All purified bacterial strains as deposited in current invention confer broad-spectrum pathogen control and resistance in plants, both in vitro and in vivo. The microbial strains impart beneficial properties in plants, in particular in agricultural plants.

Preferably, the purified bacterial strains inhibit the growth of the pathogen in the plant which has been infected with said pathogen for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%. The severity of the disease in the plant caused by the phytopathogen is highly reduced when treated with one or more of the purified bacterial strains in reference to the non-treated plant in the reference environment.

Without wishing to be bound by theory, said inhibition of growth of the pathogen may be (at least partly) linked to the expression of said polyene and/or said previously unknown compound having the formula C21H23NO4.

A second aspect of current invention concerns a bacterial population for conferring a broad-spectrum pathogen control or resistance in a plant comprising purified bacterial strains according to the invention.

The bacterial population of current invention comprises two or more purified bacterial strains. Preferably, the bacterial population comprises at least two purified bacterial strains, more preferably at least three, at least four or at least five purified bacterial strains. The purified bacterial strains of current invention in the bacterial population, each independently, contribute to the control of pathogen or pathogen resistance in plants. The combination of two or more microbial strains forms a consortia or consortium. The terms “bacterial population”, “consortia” and “consortium” are utilized interchangeably.

Preferably, the purified bacterial strains in said bacterial population are present in about equal amounts. Preferably, the concentration of each purified bacterial strain in said bacterial population is at least 10\hat{ }3 CFU or spores, at least 10\hat{ }4 CFU or spores, at least 10\hat{ }CFU or spores, at least 10\hat{ }6 CFU or spores, at least 10∂CFU or spores, at least 10\hat{ }8 CFU or spores, at least 10\hat{ }9 CFU or spores, or at least 10\hat{ }10 CFU or spores. More preferably, the concentration of each purified bacterial strain in said bacterial population is between 10\hat{ }3 to 10\hat{ }10 CFU or spores, between 10\hat{ }4 to 10\hat{ }10 CFU or spores, between 10\hat{ }5 to 10\hat{ }10 CFU or spores, between 10\hat{ }6 to 10\hat{ }10 CFU or spores, between 10\hat{ }6 to 10\hat{ }9 CFU or spores, between 10\hat{ }7 to 10\hat{ }9 CFU or spores, or between 10\hat{ }8 to 10\hat{ }9 CFU or spores.

In a preferred embodiment, the purified bacterial strain of current invention is combined with one or more other microbial strains. These other microbial strains have the ability to impart one or more beneficial properties to plant species, like for example increased pathogen resistance, increased growth, increased yield. The ability to impart these beneficial properties upon a plant is not possessed, in some embodiments, by the individual microbial strains as they would occur in nature.

Rather, in some embodiments, it is by the hand of man combining these microbial strains into a bacterial population a functional composition is developed, said functional composition possessing attributes and functional properties, which does not exist in nature.

In some embodiments, the invention is directed to synergistic combinations or mixtures of microbial species.

However, in other embodiments, the invention, as previously discussed, provides for individual isolated and biologically pure microbial strains that are able to impart beneficial properties upon a desired plant species, without the need to combine said microbial strains into consortia.

The purified bacterial strain is capable of colonizing a plant part, a plant, or a substrate comprising or hosting said plant part or plant. Successful colonization can be confirmed by detecting the presence of the strain in or near basal and/or aerial parts of the plant. For example, after spraying strains or an extract of said strains in sprayable form on leave or aerial parts of plant, the strain can be detected in or on the surface of both roots and shoots. In another example after applying the strain to the plant part, like a seed, the strain can be detected in the roots and shoots of the plants that germinate from said seed Detecting the presence of the strain inside the plant can be accomplished by measuring the viability of the strain after surface sterilization of the plant, as microbial colonization results in an internal localization of the microbial strain, rendering it resistant to conditions of surface sterilization.

The presence and quantity of the purified bacterial strain can also be established using other means known in the art, for example, immunofluorescence microscopy using microbe-specific antibodies, or fluorescence in situ hybridization. Alternatively, specific nucleic acid probes recognizing conserved sequences from a bacterial strain can be employed to amplify a region, for example by quantitative PCR, and correlated to CFUs by means of a standard curve.

In some embodiments, a modified microbial strain, plant, or plant element comprises a transgene.

In an embodiment, the purified bacterial strains are modified. These modified bacterial strains comprise a reporter gene. Examples of reporter genes encode luciferase, (green/red) fluorescent protein and variants thereof, like EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase, and the like. Preferably the reporter gene is a fluorescent gene, such as GFP. For example, GFP can be from Aequorea victoria. Other mutated forms of GFP include, but are not limited to, pRSGFP, EGFP, RFP/DsRed, and EYFP, BFP, YFP, among others, are commercially available. These bacterial strains can be used for detection. These bacterial strains can be also used for quantification assays. And these modified bacterial strains can be used for tracking purposes.

In one embodiment, the purified bacterial strain can be cultured on a culture medium or can be adapted to culture on the culture medium. Said culture medium is sterile prior to being inoculated with said bacterial strain. The medium comprises all nutrients for growth and maintenance of the strain on the culture medium. Medium for growing the bacterial strains includes a carbon source, a nitrogen source, and inorganic salts, as well as specially required substances such as vitamins, amino acids, nucleic acids and the like. In addition, the culture medium can be in a solid, semi-solid or liquid form. For example, for isolated bacteria of the disclosure, biologically pure isolates can be obtained through repeated subculture of biological samples, each subculture followed by streaking onto solid media to obtain individual colonies. Methods of preparing, thawing, and growing bacteria are commonly known.

The isolation, identification, and culturing of the microbial strains of present invention can be effected using standard microbiological techniques. Isolation can be effected by streaking the specimen on a solid medium (e.g., nutrient agar plates) to obtain a single colony, which is characterized by the phenotypic traits (e.g., Gram-positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

A following aspect of current invention relates to a microbial active ingredient for conferring a broad-spectrum pathogen control or resistance in a plant, wherein said microbial active ingredient is derived from said purified bacterial strain or bacterial population according to the invention.

Bacterial strains produce a plethora of small compounds and secondary metabolites that can be secreted in the culture or be stored endogenously. Therefore, in a particular embodiment, a supernatant from the culture wherein the bacterial strain or bacterial population of current invention has been cultured is utilized. In another embodiment, an extract or extract fraction from the culture wherein the bacterial strain or bacterial population of current invention has been cultured is useful for controlling plant pathogen or pathogen resistance. Non-limiting examples of endogenous products are amino acids, peptides, enzymes, secondary metabolites, vitamins, minerals. In some embodiments, a metabolite produced by the purified bacterial strain of the present invention is contemplated. In some embodiments, a cell-free or inactivated preparation of the purified bacterial strain of the present invention is contemplated. Removing the cell walls and/or cell membranes of the bacterial strain in culture can be obtained by several procedures which are well-known by the person skilled in the art. Non-limiting examples are the addition of chemicals to said culture, heating said culture or induce lysis in a mechanical way.

An extract can also be obtained by autolysis of the bacterial strain. In a preferred embodiment, the microbial active ingredient comprises a spore suspension, spray dried spores, or whole cell broth.

In an embodiment, the microbial active ingredient for conferring a broad-spectrum pathogen control or resistance in a plant, comprises a polyene for which a T1PKS is involved in the production pathway of said polyene, wherein said T1PKS is encoded by a gene sequence with at least 80% sequence identity to SEQ ID No 3.

In another or further embodiment, the microbial active ingredient for conferring a broad-spectrum pathogen control or resistance in a plant is a polyene macrolide, preferably Takanawaene, more preferably Takanawaene A, B or C, most preferably Takanawaene C.

In another or further embodiment, the microbial active ingredient for conferring a broad-spectrum pathogen control or resistance in a plant is a compound having the formula C21H23NO4.

In an aspect, the invention relates to the use of a microbial active ingredient as defined in any of the embodiments, for controlling a pathogen in a plant. Preferably, said plant is a crop.

In an embodiment, the pathogen for the control of which the microbial active ingredient is used, is chosen from Fusarium spp., Phytophthora spp., Pythium spp., Rhizoctonia spp., Puccinia spp., Mycosphaerella spp., Sclerotinia spp., Botrytis spp., Rhizobium spp., Colletotrichum spp., Microdochium spp., Gaeumannomyces spp., Tapesia spp., Ustilago spp., Ramularia spp., Zymospetoria spp.; preferably the pathogen is Fusarium spp, Zymospetoria spp. and/or Puccinia spp..

In an embodiment, said microbial active ingredient is derived or obtained from a purified bacterial strain as defined in any of the embodiments, or from a bacterial population as defined in any of the embodiments.

To administer the purified bacterial strain, bacterial population or microbial active ingredient to the plant part, plant, or substrate comprising or hosting said plant part, or plant it is advisable to formulate the bacterial strain, bacterial population, or microbial active ingredient in a formulation or composition. Said formulation or composition preferably comprises other compounds, such as but not limited to biologicals or agrochemicals, which for instance stimulate growth. The formulation or composition comprising said purified bacterial strain, bacterial population or microbial active ingredient is formulated to be agriculturally acceptable and can be utilized to impart one or more beneficial properties in plants, in particular to control pathogen or to confer pathogen resistance.

Another aspect of current invention relates to a formulation, preferable an agricultural active formulation, for conferring a broad-spectrum pathogen control or resistance to a plant, wherein, said formulation comprises said purified bacterial strains, said bacterial population, or said microbial active ingredient according to the invention.

Non-limiting examples of said formulation are soluble powders, soluble granules, wettable powders, tablet formulations, dry flowables, aqueous flowables, wettable dispersible granules, oil dispersions, suspension concentrates, dispersible concentrates, emulsifiable concentrates, aqueous suspensions, a fertilizer granule, or a sprayable.

In an embodiment said formulation comprises at least one oil, surfactant and polymer. Preferably, said formulation further comprises one or more of the following: fungicide, nematicide, bactericide, insecticide, molluscicide, algicide, herbicide, fertilizer, plant growth regulator, micronutrient fertilizer material, stabilizer, preservative, carrier, complexing agent, or any combination thereof. In an embodiment, the bacterial strain of the formulation is shelf-stable, and said formulation is shelf-stable. Optionally, the shelf-stable formulation is in a dry formulation, a powder formulation, or a lyophilized formulation. In some embodiments, the formulation is formulated to provide stability for the bacteria. In one embodiment, the formulation is substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years.

In another embodiment, the formulation is substantially stable at temperatures between about 4° C. and about 37° C. for at least about 5, 10, 15, 20, 25, 30 or more than 30 days.

In another embodiment, said formulation may be supplemented with secondary active ingredients. Said secondary active ingredients may include other microorganisms which are shown to elicit a beneficiary action to a plant, e.g. pathogen control but also a positive impact on plant growth and/or plant yield.

Without wishing to be limitative, said formulation may further comprise a fungal inoculant, said fungal inoculant can comprise a fungal inoculant of the family Glomeraceae, a fungal inoculant of the family Claroidoglomeraceae, a fungal inoculant of the family Acaulosporaceae, a fungal inoculant of the family Sacculospraceae, a fungal inoculant of the family Entrophosporaceae, a fungal inoculant of the family Pacidsproraceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporacea, a fungal inoculant of the family Scutellosproaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, a fungal inoculant of the genus Glomus or a combination thereof.

The bacterial inoculant can include, but is not limited to, a bacterial inoculant of the genus Rhizobium, bacterial inoculant of the genus Bradyrhizobium, bacterial inoculant of the genus Mesorhizobium, bacterial inoculant of the genus Azorhizobium, bacterial inoculant of the genus Allorhizobium, bacterial inoculant of the genus Burkholderia, bacterial inoculant of the genus Sinorhizobium, bacterial inoculant of the genus Kluyvera, bacterial inoculant of the genus Azotobacter, bacterial inoculant of the genus Pseudomonas, bacterial inoculant of the genus Azosprillium, bacterial inoculant of the genus Bacillus, bacterial inoculant of the genus Streptomyces, bacterial inoculant of the genus Paenibacillus, bacterial inoculant of the genus Paracoccus, bacterial inoculant of the genus Enterobacter, bacterial inoculant of the genus Alcaligenes, bacterial inoculant of the genus Mycobacterium, bacterial inoculant of the genus Trichoderma, bacterial inoculant of the genus Gliocladium, bacterial inoculant of the genus Klebsiella, or a combination thereof.

Said formulation may be further supplemented with a compound such as a fertilizer, a micronutrient fertilizer material, an insecticide, an herbicide, a plant growth regulator, a fungicide, a molluscicide, an algicide, a bacterial inoculant, a fungal inoculant, or a combination thereof. In some cases the fertilizer is a liquid fertilizer.

Liquid fertilizer can include without limitation, ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, hampene (chelated iron), dolomitic limestone, hydrate lime, calcium carbonate, diammonium phosphate, monoammonium phosphate, potassium nitrate, potassium bicarbonate, monopotassium phosphate, magnesium nitrate, magnesium sulfate, potassium sulfate, potassium chloride, sodium nitrates, magnesian limestone, magnesia, disodium dihydromolybdate, cobalt chlorid hexahydrate, nickel chloride hexahydrate, indole butyric acid, L-tryptophan, urea, urea-formaldehydes, urea ammonium nitrate, sulfur-coated urea, polymer-coated urea, isobutylidene diurea, K₂SO₄-2MgSO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, seabird guano, peat moss, compost, green sand, cottonseed meal, feather meal, crab meal, fish emulsion or a combination thereof. The micronutrient fertilizer material can comprise boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate or a combination thereof. The insecticide can include an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof. The herbicide can comprise a chlorophenoxy compound, a nitrophenolic compound, a nitrocresolic compound, a dipyridyl compound, an acetamide, an aliphatic acide, an anilide, a benzamide, a benzoic acid, a benzoic acid derivative, anisic acid, an anisic acid derivative, a benzonitrile, benzothiadiazinone dioxide, a thiocarbamate, a carmabate, carbanilate, chloropyridinyl, a cyclohexenone derivative, a dinitroaminobenzene derivative, a fluorodinitrotoluidine compound, isoxazolidinone, nicotinic acide, isopropylamine, an isopropulamine derivative, oxadiazolinone, a phosphate, a phthalate, a picolinic acid compound, a triazine, a triazole, a uracil, a urea derivative, endothall, sodium chlorate, or a combination thereof. The fungicide can comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof.

In a more preferred embodiment of current invention said formulation further comprises an agriculturally compatible carrier. Said “agriculturally compatible carrier” which can be regarded as a vehicle, is generally inert and it must be acceptable in agriculture. Thus, the phrase “agriculturally compatible” denotes a substance that can be used routinely under field conditions without interfering with growers' planting equipment, and without adversely influencing crop development or the desired ecological balance in a cultivated area.

The agriculturally compatible carrier can be solid. Solid carriers can include but are not limited to clays, natural or synthetic silicates, silica, resins, waxes, solid fertilizers, a polymer, a granular mass, perlite, a perlite granule, peat, a peat pellet, soil, vermiculite, charcoal, sugar factory carbonation press mud, rice husk, carboxymethyl cellulose, fine sand, calcium carbonate, flour, alum, a starch, talc, polyvinyl pyrrolidone, or a combination thereof. The agriculturally compatible carrier can be a liquid. Liquid carriers can include but are not limited to water, alcohols, ketones, petroleum fractions, oils, aromatic or paraffinic hydrocarbons, chlorinated hydrocarbons, liquefied gases or a combination thereof. More particularly, the agriculturally compatible carrier can include a dispersant, a surfactant, an additive, a thickener, an anti-caking agent, residue breakdown, a composting formulation, a granular application, diatomaceous earth, a coloring agent, a stabilizer, a preservative, a polymer, a coating or a combination thereof. One of the ordinary skills in the art can readily determine the appropriate carrier to be used taking into consideration factors such as a particular bacterial strain, plant to which the inoculum is to be applied, type of soil, climate conditions, whether the inoculum is in liquid, solid or powder form, and the like. The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof. The proteinaceous material can include a milk product, wheat flour, soybean meal, blood, albumin, gelatin, or a combination thereof. The thickener can comprise a long chain alkylsulfonate of polyethylene glycol, polyoxyethylene oleate or a combination thereof. The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination thereof. The anti-caking agent can include a sodium salt such as a sodium sulfite, a sodium sulfate, a sodium salt of monomethyl naphthalene sulfonate, or a combination thereof; or a calcium salt such as calcium carbonate, diatomaceous earth, or a combination thereof. The agriculturally compatible carrier can also include a fertilizer, a micronutrient fertilizer material, an insecticide, a herbicide, a plant growth amendment, a fungicide, a molluscicide, an algicide, a bacterial inoculant, a fungal inoculant, or a combination thereof. Non-limiting examples are provided above.

Preferably, the formulation of current invention comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

In a preferred embodiment said bacterial strains or bacterial population are present in spray-dried or freeze-dried form.

The formulation herein are shelf-stable. In some aspects, the microbial strains are spray-dried or freeze-dried. Drying processes for microorganisms are known for a person skilled in the art. In some aspects, the formulation has adjuvants that provide for a pertinent shelf life. Also described herein are a plurality of purified bacterial strains confined within an object selected from the group consisting of: bottle, jar, ampule, package, vessel, bag, box, bin, envelope, carton, container, silo, shipping container, truck bed, and case.

Current invention also relates to the formulations being formulated to provide a high colony forming units (CFU) of purified bacterial strains and consortia comprising the same. Preferably, the CFU concentration of the taught formulations is higher than the concentration at which the microbial strains would exist naturally.

In a more preferred embodiment of said formulation, said purified bacterial strain is present at a CFU concentration of at least 10′CFU/ml when in a liquid formulation or 10′CFU/g when in a non-liquid formulation.

Preferably, said purified bacterial strain is present at a CFU concentration of at least 1×10\hat{ }4 CFU, at least 2×10\hat{ }4 CFU, at least 3×10\hat{ }4 CFU, at least 4×10\hat{ }4 CFU, at least 5×10\hat{ }4 CFU, at least 6×10\hat{ }4 CFU, at least 7×10\hat{ }4 CFU, at least 8×10\hat{ }4 CFU, at least 9×10\hat{ }4 CFU, at least 10×10\hat{ }4 CFU, at least 2×10\hat{ }5 CFU, at least 3×10\hat{ }5 CFU, at least 4×10\hat{ }5 CFU, at least 5×10\hat{ }5 CFU, at least 6×10\hat{ }5 CFU, at least 7×10\hat{ }5 CFU, at least 8×10\hat{ }5 CFU, at least 9×10\hat{ }5 CFU, at least 10×10\hat{ }5 CFU, at least 2×10\hat{ }6 CFU, at least 3×10\hat{ }6 CFU, at least 4×10\hat{ }6 CFU, at least 5×10\hat{ }6 CFU, at least 6×10\hat{ }6 CFU, at least 7×10\hat{ }6 CFU, at least 8×10\hat{ }6 CFU, at least 9×10\hat{ }6 CFU, at least 10×10\hat{ }6 CFU, at least 2×10\hat{ }7 CFU, at least 3×10\hat{ }7 CFU, at least 4×10\hat{ }7 CFU, at least 5×10\hat{ }7 CFU, at least 6×10\hat{ }7 CFU, at least 7×10\hat{ }7 CFU, at least 8×10\hat{ }7 CFU, at least 9×10\hat{ }7 CFU, at least 10×10\hat{ }7 CFU, at least 2×10\hat{ }8 CFU, at least 3×10\hat{ }8 CFU, at least 4×10\hat{ }8 CFU, at least 5×10\hat{ }8 CFU, at least 6×10\hat{ }8 CFU, at least 7×10\hat{ }8 CFU, at least 8×10\hat{ }8 CFU, at least 9×10\hat{ }8 CFU, at least 10×10\hat{ }8 CFU, at least 2×10\hat{ }9 CFU, at least 3×10\hat{ }9 CFU, at least 4×10\hat{ }9 CFU, at least 5×10\hat{ }9 CFU, at least 6×10\hat{ }9 CFU, at least 7×10\hat{ }9 CFU, at least 8×10\hat{ }9 CFU, at least 9×10\hat{ }9 CFU, at least 10×10\hat{ }9 CFU per ml when in a liquid formation or per gram when in a non-liquid formation.

More preferably, said purified bacterial strain is present at a CFU concentration of between 10×10\hat{ }4 and 10×10\hat{ }9 CFU, between 2×10\hat{ }5 and 9×10\hat{ }9 CFU, between 3×10\hat{ }5 and 8×10\hat{ }9 CFU, between 4×10\hat{ }5 and 7×10\hat{ }9 CFU, between 5×10\hat{ }5 and 6×10\hat{ }9 CFU, between 6×10\hat{ }5 and 5×10\hat{ }9 CFU, between 7×10\hat{ }5 and 4×10\hat{ }9 CFU, between 8×10\hat{ }5 and 3×10\hat{ }9 CFU, between 9×10\hat{ }5 and 2×10\hat{ }9 CFU, between 10×10\hat{ }5 and 10×10\hat{ }8 CFU. More preferably between 2×10\hat{ }6 and 10×10\hat{ }8 CFU, between 3×10\hat{ }6 and 10×10\hat{ }8 CFU, between 4×10\hat{ }6 and 10×10\hat{ }8 CFU, between 5×10\hat{ }6 and 10×10\hat{ }8 CFU, between 6×10\hat{ }6 and 10×10\hat{ }8 CFU, between 7×10\hat{ }6 and 10×10\hat{ }8 CFU, between 8×10\hat{ }6 and 10×10\hat{ }8 CFU, between 9×10\hat{ }6 and 10×10\hat{ }8 CFU, between 10×10\hat{ }6 and 10×10\hat{ }8 CFU per ml when in a liquid formation or per gram when in a non-liquid formation. Even more preferably between 2×10\hat{ }7 and 10×10\hat{ }8 CFU, between 3×10\hat{ }7 and 10×10\hat{ }8 CFU, between 4×10\hat{ }7 and 10×10\hat{ }8 CFU, between 5×10\hat{ }7 and 10×10\hat{ }8 CFU, between 6×10\hat{ }7 and 10×10\hat{ }8 CFU, between 7×10\hat{ }7 and 10×10\hat{ }8 CFU, between 8×10\hat{ }7 and 10×10\hat{ }8 CFU, between 9×10\hat{ }7 and 10×10\hat{ }8 CFU per ml when in a liquid formation or per gram when in a non-liquid formation.

In a preferred embodiment, said formulation is formulated to a high colony forming units (CFU) of purified bacterial strain, or bacterial population. The CFU concentration of the formulation is higher than the concentration at which the bacteria would exist naturally. Said CFU concentration is effective to impart the control pathogen and resistance in plants.

In an embodiment, said formulation is heterologously disposed, for example, on the surface of the plant, plant part or substrate comprising or hosting said plant or plant part, in an amount effective to be detectable in the plant itself, the plant grown from the plant part, or plant grown in said substrate and subsequent offspring. In a particular embodiment, the purified bacterial strains is heterologously disposed in an amount effective to be detectable in an amount of at least about 100 CFU or spores, between 100 and 200 CFU or spores, at least about 200 CFU or spores, between 200 and 300 CFU or spores, at least about 300 CFU or spores, between 300 and 400 CFU or spores, at least about 500 CFU or spores, between 500 and 1,000 CFU or spores, at least about 1,000 CFU or spores, between 1,000 and 3,000 CFU or spores, at least about 3,000 CFU or spores, between 3,000 and 10,000 CFU or spores, at least about 10\hat{ }4 CFU or spores, between 10\hat{ }4 and 30,000 CFU or spores, at least about 30,000 CFU or spores, between 30,000 and 10\hat{ }5 CFU or spores, at least about 10\hat{ }5 CFU or spores, between 10\hat{ }5 and 10\hat{ }6 CFU or spores, at least about 10\hat{ }6 CFU or spores, or more, in and/or on the plant, plant part or substrate comprising said plant or plant part. The purified bacterial strains may also be detectable in an amount as listed above in the subsequent offspring of the plant, or plant part which has been heterologously disposed with said strains.

The formulation of the present invention can be applied to the soil, plant, seed, rhizosphere, or other areas of the plant to which it would be beneficial to apply the formulation comprising the purified bacterial strain, bacterial population or microbial active ingredient as described above.

A further aspect of current invention also concerns a plant part, such as a shoot, leaves, or a seed, comprising the formulation according to current invention. The purified bacterial strains or microbial consortia may be applied on a plant part as a spray and/or as a coating. In a further embodiment, said plant part comprising said purified bacterial strains, microbial consortia or formulations according to the current invention comprises an amount of bacteria larger than a possible amount already inherently present on or in said plant part. Said amount is at least 10 times, 50 times, 100 times, 500 times, 1000 times larger than a possible amount already present on or in said surface.

In one embodiment, the microorganisms are applied in the form of coatings or other application. In embodiments, the coating may be applied to a naked and untreated plant part. In other embodiments, the coating may be applied as an overcoat to a previously treated plant part. Preferably, the microorganisms are applied in the form of seed coatings or other applications to the seed. Seed coatings are particularly preferred in the treatment of soil borne fungal diseases. In embodiments, the seed coating may be applied to a naked and untreated seed. In other embodiments, the seed coating may be applied as a seed overcoat to a previously treated seed. Applying said bacterial strains, microbial consortia or formulation to plant parts, like a seed, the plant itself or its substrate modulates a trait of agronomic importance. The trait of agronomic importance can be amongst others pathogen resistance, plant growth, and/or plant yield.

In another embodiment, the microorganisms, consortia or formulations as disclosed herein are added onto a surface, such as a soil surface, or plant surface. In a further embodiment, said purified bacterial strains, the microbial consortia or formulations as disclosed herein may be applied on a plant part surface or soil surface by means of spraying. To that purpose, said purified bacterial strains, microbial consortia or formulations are formulated to allow spraying. In a further embodiment, said purified bacterial strains, microbial consortia or formulations according to the current invention are formulated such that the amount of bacteria added is larger than a possible amount already inherently present on or in said surface. Said amount is at least 10 times, 50 times, 100 times, 500 times, 1000 times larger than a possible amount already present on or in said surface.

In a more specific embodiment, the microorganisms are applied in the form of sprayable forms including but not limited to live cells, spores, spray-dried cells, spray-dried spores, freeze-dried cells and/or freeze-dried spores.

In another embodiment, the microorganisms are applied in the form of fresh cells and/or fresh spores.

In current invention the plant part is effectively treated against the plant pathogen by conferring the pathogen control and resistance the plant, by coating said plant part with purified bacterial strains or consortia in an amount that is not normally found on the plant part. This also applies for a plant itself or the substrate comprising or hosting the plant or plant part.

In current invention the plant part is also effectively treated against the plant pathogen by conferring the pathogen control and resistance the plant, by spraying said plant part with purified bacterial strains or consortia in an amount that is not normally found on the plant part. This also applies for a plant itself or the substrate comprising or hosting the plant or plant part. A non-limiting example concerns the spraying of one or more purified bacterial strains or bacterial consortia of current invention against Fusarium spp. by spraying the ears of a Triticum aestivum plants. A curative or preventive treatment of Triticum aestivum plants against Puccinia spp. infections may for example be attributed by spraying the agricultural formulation of current invention on the leaves of said plants. The biocontrol of Botrytis spp. in strawberry (Fragaria species) and tomato (Solanum lycopersicum) is for example established via foliar application of purified bacterial strains of current invention. Also for leafy vegetables, like for instance Lactuca spp., the crop may be treated against Rhizoctonia spp. and/or Pythium spp. by spraying purified bacterial strains of current invention on the leaves of said crop.

In some embodiments, the applied bacteria may become endophytic and consequently will be present in the growing plant, or plant part that was treated and its subsequent offspring.

Another aspect relates to the use of the purified bacterial strain according to the current invention, the bacterial population according to the current invention, the microbial active ingredient and/or the formulation according to current invention to control a pathogen in a plant, wherein said plant is preferably a crop. In an embodiment, the pathogen for the control of which the purified bacterial strain, the bacterial population, the microbial active ingredient and/or the formulation is used, is chosen from Fusarium spp., Phytophthora spp., Pythium spp., Rhizoctonia spp., Puccinia spp., Mycosphaerella spp., Sclerotinia spp., Botrytis spp., Rhizobium spp., Colletotrichum spp., Microdochium spp., Gaeumannomyces spp., Tapesia spp., Ustilago spp., Ramularia spp., Zymospetoria spp.; preferably the pathogen is Fusarium spp., Zymospetoria spp. and/or Puccinia spp..

In some embodiments, the purified bacterial strains as described above in any of the embodiments can be utilized, in a method of imparting one or more beneficial properties or traits to a desired plant species.

In some embodiments, the bacterial population or consortium of any of the embodiments can be utilized, in a method of imparting one or more beneficial properties or traits to a desired plant species.

In some embodiments, the microbial active ingredient of any of the embodiments can be utilized, in a method of imparting one or more beneficial properties or traits to a desired plant species.

In some embodiments, the agriculturally acceptable composition containing the purified bacterial strains, bacterial population or consortium, or microbial active ingredient of current invention can be utilized, in a method of imparting one or more beneficial properties or traits to a desired plant species.

A further aspect of current invention relates to a method for controlling a pathogen in a plant, which comprises artificially introducing a purified bacterial strain, a bacterial population or a formulation comprising purified bacterial strains, bacterial population, or microbial active ingredient according to current invention to a plant, a plant part or a substrate comprising or hosting said plant or plant part, thereby conferring a broad-spectrum pathogen control or resistance to a plant.

The method is applicable in a wide range of agricultural applications. The method of current invention also controls the pathogen resistance in a plant.

Inoculating the plant, plant part or substrate with the microorganism confers a broad-spectrum pathogen control or resistance in a plant. Furthermore, said treatment effectively inhibits or reduces pathogen infection. As a result the pathogen can be prevented in treated plants. And if plants are infected with the pathogen, in case said plant was not treated against the pathogen or was wrongly treated against the pathogen, said treatment results in a growth inhibition of the pathogen. Despite the pathogen, the plant growth and/or yield can be preserved or even be improved in comparison to a reference plant in a reference environment.

Alternatively or in addition, the purified bacterial strains or consortia of current invention may be present in the formulation in an amount effective to increase the biomass and/or yield of a plant that has had such formulation applied thereto, by at least 1%, at least 2%, at least 3%, at least 5%, at least 10%, at least 15%, at least 20%, at least 30%, at least 40%, at least 50%, at least 60%, at least 70%, at least 80%, at least 90%, at least 100%, or more, when compared with the reference plant that has not had the formulations applied.

Alternatively or in addition, the purified bacterial strains or consortia as described above may be present in the formulation in an amount effective to detectably modulate an agronomic trait of interest of a plant that has had such a formulation applied thereto, by at least 1%, at least 2%, at least 3%, at least 5%, at least 10%, at least 15%, at least 20%, at least 30%, at least 40%, at least 50%, at least 60%, at least 70%, at least 80%, at least 90%, at least 100%, or more, when compared with the reference plant that has not had the formulations applied.

In a preferred embodiment of said pathogen is selected from the group consisting of Fusarium spp. (F. graminearum, F. pseudograminearum, F. oxysporum), Phytophthora spp. (e.g. P. infestans), Pythium spp., Rhizoctonia spp. (e.g. R. solani), Puccinia spp., Mycosphaerella spp. (e.g. M. graminicola also named Zymoseptoria tritici), Sclerotinia spp. (e.g. S. sclerotiorum, S. minor, S. trifoliorum), Botrytis spp. (e.g. B. cinerea), Rhizobium spp., Colletotrichum spp. (e.g. C. graminicola), Microdochium spp. (e.g. M. nivale), Gaeumannomyces spp. (e.g. G. graminis), Tapesia spp. (e.g. T. yallundae), Ustilago spp., Ramularia spp. (R. collo-cygni), Zymospetoria spp. (e.g. Z. tritici).

Above listed pathogens are the causal agent of known plant diseases. Alternatively, the plant disease is a combination of different phytopathogenic fungi infections as listed above herein. Therefore, the method of current invention also effectively controls one or more pathogens in plants.

The current method and means are particularly useful to be used for monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Axonopus spp., Bambusa spp., Benincasa hispida, Bertholletia excelsea, Beta vulgaris, Brassica spp. (e.g. Brassica napus), Bouteloua spp., Brassica spp., Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Coix spp., Colocasia esculenta, Cola spp., Corchorus sp., Coriandrum sativum, Corylus spp., Crataegus spp., Crocus sativus, Cucurbita spp., Cucumis spp., Cynara spp., Cynodon spp., Dactylis spp., Daucus carota, Desmodium spp., Dimocarpus longan, Dioscorea spp., Diospyros spp., Echinochloa spp., Elaeis spp. (e.g. Elaeis guineensis, Elaeis oleifera), Eleusine coracana, Eragrostis spp. (e.g. Eragrostis tef), Eremochloa spp., Erianthus spp., Eriobotrya japonica, Eucalyptus spp., Eugenia uniflora, Fagopyrum spp., Fagus spp., Festuca spp. (e.g. Festuca arundinacea), Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca spp. (e.g. Lactuca sativa), Lathyrus spp., Lens culinaris, Linum usitatissimum, Litchi chinensis, Lolium spp., Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Paspalum spp., Panicum spp. (e.g. Panicum miliaceum, Panicum virgatum), Passiflora edulis, Pastinaca sativa, Pennisetum spp., Persea spp., Petroselinum crispum, Phalaris arundinacea, Phaseolus spp., Phleum spp. (e.g. Phleum pretense), Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale spp. (e.g. Secale cereal), Sesamum spp., Sinapis sp., Solanum spp. (e.g. Solanum tuberosum, Solanum integrifolium, Solanum lycopersicum), Sorghum spp. (e.g. Sorghum bicolor), Spinacia spp., Stenotaphrum spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum, Triticum vulgare), Topaeolum spp. (e.g. Tropaeolum minus, Tropaeolum majus), Vaccinium spp., Vicia spp., Vigna spp., Viola odorata, Vitis spp., Zea mays, Zizania palustris, Ziziphus spp., Zoysia spp., amongst others; including the progenies and hybrids between the above.

In a particular embodiment, the plant of current invention is selected from Triticum spp., Secale spp., Hordeum spp., Solanum spp., Fragaria spp., or Lactuca spp.

In a more preferred embodiment, it is contemplated that the plant, more in particular the agricultural plant, of the present invention is wheat (Triticum aestivum and related varieties), rye (Secale cereale and related varieties) barley (Hordeum vulgare and related varieties), tomato (Solanum lycopersicum and related varieties, strawberry (Fragaria species and related varieties), or lettuce (Lactuca sativa and related varieties).

In another particular embodiment, the current invention discloses a novel method of discovering antifungal compounds in bacterial strains. This method includes the high-throughput screening of microbial strain extracts or fractionations thereof for their antifungal activity against specific phytopathogenic fungi of interest.

The yet another particular embodiment, the current invention discloses a method for discovering the anti-pathogenic compounds of the microbiological strains, wherein the methods consist of mass spectrometry analysis of extractions of the strains and in silico analysis of whole Genome Sequences (WGS) data.

In another embodiment, the current invention discloses that antifungal activity of B/00227, B/00225, B/00228, B/00226 and B/00231 is caused by a type of polyene macrolide, whereby the polyene ring contains 5 double bonds (pentaene macrolide).

More particularly, the current invention discloses that the purified strain B/00227, B/00225, B/00228, B/00226 and B/00231 specifically produces a pentaene macrolide compound that was identified as a Takanawaene.

More precisely, the current invention discloses that the purified strain B/00228, B/00227, B/00225, B/00226 and B/00231 produces Takanawaene C.

In yet more particular embodiment the current invention discloses that antifungal activity of B/00227, B/00225, B/00228, B/00226 and B/00231 is caused by Takanawaene C.

In a more particular embodiment the current invention discloses that a Type 1 Polyketide Synthase (T1PKS) is involved in the production pathway of Takanawaene C. In particular, said T1PKS is encoded by a gene sequence with at least 75%, preferably at least 77%, more preferably at least 80% sequence identity to SEQ ID No 3.

It will be understood by a person skilled in the art that the invention also relates to the use of a purified bacterial strain producing Takanawaene A, B or C, preferably Takanawaene C in crop protection.

In addition, the invention also relates to the use of Takanawaene A, B or C, preferably Takanawaene C as biocontrol agent against plant pathogens. Said plant pathogens may be any of the pathogens as described above. Preferably said plant pathogen is Fusarium spp., Zymospetoria spp. and/or Puccinia spp..

In another or further embodiment, the current invention discloses that the purified strain B/00228, B/00227, B/00225, B/00226 and B/00231 produce/express a compound having the formula C21H23NO4. Said compound was previously unknown, and not disclosed in the Natural Products Databases. In addition, said compound has been shown to have antibacterial and antifungal activity against one or more pathogens, preferably plant pathogens.

It will be understood by a person skilled in the art that the invention also relates to such compound having the formula C21H23NO4. In a preferred embodiment, said compound is capable of controlling a pathogen in a plant, preferably in a crop.

In an embodiment of the invention, said compound having the formula C21H23NO4 is obtained from bacteria or is chemically synthesized.

In a further embodiment, said compound is obtained from a purified bacterial strain, or bacterial population as described before in any of the embodiments, in particular form any of strains B/00228, B/00227, B/00225, B/00226 and B/00231.

The invention also relates to such compound having the formula C21H23NO4 for use as an antimicrobial and/or antifungal agent.

The present invention also relates to the use of said compound, strain or population producing said compound, for use in crop protection, and/or for use in the treatment of bacterial and/or fungal infections in a plant.

In addition, the invention also relates to the use of a compound having the formula C21H23NO4 as biocontrol agent, preferably against plant pathogens.

Said plant pathogens may be any of the pathogens as described above. Preferably said plant pathogen is Fusarium spp., Zymospetoria spp. and/or Puccinia spp..

Inoculating the substrate comprising or hosting said plant or plant part can be performed, by way of example and without the intention to be limiting, using a powder, a granule, a pellet, a plug, or a soil drench that is applied to the substrate.

Inoculation could also be performed by a liquid application, such as a foliar spray or liquid composition. The application may be applied to a growing plant or to the substrate. Plants, in particular agricultural plants, can be grown in substrate. In one embodiment, said substrate is soil, sand, gravel, polysaccharide, mulch, compost, peat moss, straw, logs, clay, or a combination thereof. In another embodiment, the substrate can also include a hydroculture system or an in vitro culture system. In some embodiments, a combination of different application methods as described herein is applied. In a non-limiting example, wheat plants are treated against Fusarium spp., Zymospetoria spp. and Puccinia spp. by foliar application. Also strawberry plants and tomato plants may for example be treated against Botrytis spp. by foliar application. In another non-limiting example, lettuce plants are treated against Rhizoctonia spp. and Pythium spp. by foliar application, subsequently followed by soil drench applications.

A final aspect of current invention concerns a method for screening a bacterial organism in order to evaluate whether said organism may confer a broad-spectrum pathogen control or resistance to a plant, said method comprises screening said organism for the presence of SEQ ID No 1 in the 16S nucleotide sequence of said organism and for the presence of SEQ ID No 3 or a gene sequence having at least 75%, preferably at least 77%, more preferably at least 80% sequence identity with SEQ ID No 3 in the genomic sequence of said organism. Preferably the method further comprises screening said organism for the presence of a nucleotide sequence having at least 95% sequence identity to SEQ ID No 2, preferably said organism has a nucleotide sequence which is identical to SEQ ID No 2 over its entire length.

Several techniques are known in the art for screening bacteria for the presence of a nucleotide sequence of interest. Several molecular techniques have been developed and have been extensively used, like for example detection methods using polymerase chain reaction (PCR)-based assays. These assays are known for a person skilled in the art.

In a preferred embodiment said method comprises screening said organism for the presence of SEQ ID No 1 in a 16S nucleotide sequence of said organism using a forward primer and a reverse primer.

Current invention provides the nucleotide sequence of the forward primer, called 907F, and the reverse primer, called 1100R, as specified in Table 1 as respectively SEQ ID No 5 and SEQ ID No 6. The forward and reverse primer amplify a defined nucleotide sequence of about 205 base pairs, which is also provided for the five Kitasatospora-like strains as deposited B/00227, B/00225, B/00228, B/00226, and B/00231, as SEQ ID No 4. The defined nucleotide sequence comprises the signature sequence of current invention.

The set of primers, the forward and reverse primer, are specific for detecting a defined sequence of the V6 region of the 16S nucleotide sequence in a bacterial strain in which SEQ ID No 1 is present.

The invention is further described by the following non-limiting examples which further illustrate the invention, and are not intended to, nor should they be interpreted to, limit the scope of the invention.

The present invention will be now described in more details, referring to examples that are not limitative.

### BACTERIAL DEPOSIT

The bacterial strains of current invention are deposited on 21 Aug. 2019 with the Polish Collection of Microorganisms (Institute of Immunology and Experimental Therapy, Polish Academy of Sciences, UI. Weigla 12, 53-114 Wroclaw, Poland), under the terms of the Budapest Treaty with Deposit ID: B/00227, B/00225, B/00228, B/00226, and B/00231.

The biological material shall be made available as provided for under Rule 13bis.6 PCT and Rule 32(1) EPC only by the issuance of a sample to an Expert.

### SEQUENCE LISTING

A sequence listing is provided containing the signature sequence (SEQ ID No 1), which contains 16 base pairs. The 16S nucleotide sequence of the Kitasatospora-like strains of current invention, of which the signature sequence is marked in bold, is provided in the sequence listing as SEQ ID No 2. The sequence listing also provides a gene sequence listed as SEQ ID No 3. The nucleotide region defined by forward primer 907F (SEQ ID No 5) and reverse primer 1100R (SEQ ID No 6) of the five Kitasatospora strains B/00227, B/00225, B/00228, B/00226, B/00231 is the V6 region provided by SEQ ID No 4. In said defined V6 region the signature sequence is present and marked in bold. The 6 sequences are listed in Table 1.

TABLE 1

| SEQ | |
|---|---|
| ID | |
| No | Nucleotide sequence (5′-3′) |
| 1 | ACCGTGGAGACACGGT |
| 2 | CACGGAGAGTTTGATCCTGGCTCAGGACGAACGCTGGCGGCGTGCTTAACACATGCAAGTCGA |
| | ACGGTGAAGCCCTTCGGGGTGGATCAGTGGCGAACGGGTGAGTAACACGTGGGCAATCTGCC |
| | CTGCACTCTGGGACAAGCCCTGGAAACGGGGTCTAATACCGGATATGACCTTCCTCCGCATGG |
| | GGGTTGGTGGAAAGCTCCGGCGGTGCAGGATGAGCCCGCGGCCTATCAGCTTGTTGGTGGGG |
| | TAATGGCCTACCAAGGCGACGACGGGTAGCCGGCCTGAGAGGGCGACCGGCCACACTGGGAC |
| | TGAGACACGGCCCAGACTCCTACGGGAGGCAGCAGTGGGGAATATTGCACAATGGGCGAAAG |
| | CCTGATGCAGCGACGCCGCGTGAGGGATGACGGCCTTCGGGTTGTAAACCTCTTTCAGCAGGG |
| | AAGAAGCGCAAGTGACGGTACCTGCAGAAGAAGCACCGGCTAACTACGTGCCAGCAGCCGCG |
| | GTAATACGTAGGGTGCGAGCGTTGTCCGGAATTATTGGGCGTAAAGAGCTCGTAGGCGGCCTG |
| | TCGCGTCGGATGTGAAAGCCCGGGGCTTAACCCCGGGTCTGCATTCGATACGGGCAGGCTAG |
| | AGTGTGGTAGGGGAGATCGGAATTCCTGGTGTAGCGGTGAAATGCGCAGATATCAGGAGGAA |
| | CACCGGTGGCGAAGGCGGATCTCTGGGCCATTACTGACGCTGAGGAGCGAAAGCGTGGGGAG |
| | CGAACAGGATTAGATACCCTGGTAGTCCACGCCGTAAACGTTGGGAACTAGGTGTTGGCGACA |
| | TTCCACGTCGTCGGTGCCGCAGCTAACGCATTAAGTTCCCCGCCTGGGGAGTACGGCCGCAAG |
| | GCTAAAACTCAAAGGAATTGACGGGGGCCCGCACAAGCAGCGGAGCATGTGGCTTAATTCGAC |
| | GCAACGCGAAGAACCTTACCAAGGCTTGACATATGCCGGAAAACCGTGGAGACACGGTCCCC |
| | CTTGTGGTCGGTATACAGGTGGTGCATGGTTGTCGTCAGCTCGTGTCGTGAGATGTTGGGTTA |
| | AGTCCCGCAACGAGCGCAACCCTTGTTCTGTGTTGCCAGCATGCCTTTCGGGGTGATGGGGAC |
| | TCACAGGAGACTGCCGGGGTCAACTCGGAGGAAGGTGGGGACGACGTCAAATCATCATGCCC |
| | CTTATGTCTTGGGCTGCACACGTGCTACAATGGTCGGTACAAAGGGCTGCGATGCCGTGAGGC |
| | GGAGCGAATCCCAAAAAGCCGGCCTCAGTTCGGATTGGGGTCTGCAACTCGACCCCATGAAGT |
| | TGGAGTTGCTAGTAATCGCAGATCAGCATGCTGCGGTGAATACGTTCCCGGGCCTTGTACACA |
| | CCGCCCGTCACGTCACGAAAGTCGGTAACACCCGAAGCCGGTGGCCTAACCCGTAAGGGGAG |
| | GAGCCGTCGAAGGTGGGACCAGCGATTGGGACGAAGTCGTAACAAGGTAGCCGTACCGGAAG |
| | GTGCGGCTGGATCACCTCCTTT |
| 3 | ATGCAGAATGAGCAGAACGAGCAGAAGCTGCTCGACTACCTCAAGCGTGTGACGGCCGATCTC |
| | AAGCAGACCCGGCGCCGGTTGCACGAGGTGGAGACCAAGGACCAGGAGCCGATCGCCATCGT |
| | GGCGATGGGCTGCCGTTACCCCGGGCAGGTCAACAGCCCCGAGGAGCTCTGGCAACTGGTCG |
| | AGCGCGGTGGTGACGCGGTCTCCGGCTTCCCCACCAACCGCGGTTGGGACCTGGAGGCGCTC |
| | TACGACCCGGACCCCGAGAGCCCCGGGAAGTGCTACACCCGCGAGGGCGGGTTCCTGCACGG |
| | GGCCGGGGAGTTCGACCCGTCGTTCTTCGGGATCTCGCCGCGCGAGGCGCTGGCCACCGACC |
| | CGCAGCAGCGGCTGCTGCTGGAGGTCGCCTGGGAGACCTTCGAGCGCGCCGGCATCGACCCC |
| | TCCTCGGTCAGGGGCTCGAACACCGGCGTCTTCGTCGGCCTCGCCTACCAGGGGTACGCGGG |
| | CGACGAGGAGAGCGCACAGGAGCTGGAGGGCTTCCTGCTGACCGGCACCGCGCCGAGCGTC |
| | GCCTCGGGCCGGCTCTCCTACACCTTCGGGCTCGAAGGCCCGGCGCTGACCGTGGACACCGC |
| | GTGCTCGTCCTCGCTGGTCGCCCTGCACCTGGCGATCCAGGCCCTGCGCCGGCGGGAGTGCA |
| | CCCTCGCGCTGGCCGGCGGTGCGGCGATCATGGCGAGCACCGGCATGTTCACCGAGTTCAGC |
| | CGCCAGCAGGGCCTCGCCGTGGACGGCCGCTGCAAGTCCTTCGCCGCCGAGGCGGACGGCAC |
| | GGGCTGGGGCGAGGGCGTCGGCATGCTGCTGGTCGAGCGGCTCTCCGATGCCGAGCGCAAC |
| | GGTCACCCGGTGCTGGCGGTGGTGCGGGGTTCGGCCGTGAACCAGGACGGCGCGTCCAACG |
| | GTCTGACGGCCCCGAACGGCCCCGCCCAGCAGCGGGTGATCCGTCAGGCGTTGGCCAGTGCG |
| | GGGCTGTCGGCGGCGGACGTGGACGTGGTCGAGGCGCACGGTACGGGGACGACGCTGGGTG |
| | ACCCGATCGAGGCCCAGGCGCTGCTGGCGACGTACGGGCAGGACCGGCCCGAGGACCGTCC |
| | GCTGTGGCTGGGCTCGTTGAAGTCCAACATCGGTCACACCCAGGCGGCGGCCGGGGGGGCG |
| | GCATCATCAAGATGGTGCAGGCGATGCGGCACGGTGTTCTGCCGCGCACCCTGCACGCGGAG |
| | CAGCCGACGCCGCACGTCGACTGGTCGGCGGGCGCGGTACGGCTGCTGACCGAGCCGGTCG |
| | CCTGGCCGGAGGGCGACCGCCCGCGGCGGGCCGCCGTCTCCTCCTTCGGGGTCAGCGGCAC |
| | CAACGCGCACACCATCATCGAGCAGGCCCCGGCCGCCCCCGAGGCGGACGAGGAGCCGCCC |
| | GCCCGGGCCGAGCCGACGCCCGCCGCGGCGCCGCTGTCCTGGGTGGTCTCCGCCCGCAGCA |
| | GGGCCGCCCTGCGCGGACAGGCGGAACGGCTGCTCTCCCAGGTGATCGGCCTGCCCGACGCG |
| | CGGCCGCTCGACGTCGCCTACTCGCTGGCGACCTCCCGTGCCGCCCTGGACCACCGCGCCGC |
| | GGTGGTCGGCACGGACCTCGCCGAGCTCATCGACGGCGTCCGCGCGCTGGCCGAGGGGAAG |
| | GCGGCCCCGCAGGTGATCCAGGGACACACCGGCCGGGACGCCGGGCTCGCCCTCGTGTTCTC |
| | GGGGCAGGGGAGCCAGCGGCTGGGGATGGGGCGTGAACTGTACGCGTCCTTCCCGGTGTTC |
| | GCCGAGGCGTTCGACGCGGTGTGTGCGGCGGTGGACGCCCACCTGGAACGCCCGCTGCGCG |
| | AGGTGGTGTTCGGCGAGGACGCGGAGCTGCTGAACCGGACGGAGTTCACCCAGCCGGCGTTG |
| | TTCGCGGTGGAGGTGGCGCTCTTCCGGCTCACCGAGTCGTGGGGGGTGCGGGCGGACTTCCT |
| | GGCCGGGCACTCGATCGGTGAGTTCGCCGCCGCCCATGTGGCGGGTGTTCTGTCGCTGGCCG |
| | ATGCGGCCGAACTGGTCGTGGCCCGTGGAAGGTTGATGCAGGAGCTGCCCTCGGGCGGTGCG |
| | ATGGTGGCGGTGCAGGCCTCCGAGGAGGAGGTGGCGCCGCTGCTCGCCGGCCGCGAGGAGC |
| | GCGTGGGTATCGCGGCGGTCAACGGCCCGTCCTCGGTGGTCATTTCGGGTGCCGAGGACGCG |
| | GTGGCGGAGGTGGTGGAGGCGCTGGCGGCGGACGGATGCCGGACGAAGCGCCTCGCGGTCT |
| | CTCACGCGTTCCACTCGCCGCTGATGGAGCCGATGCTCGCGGCCTTCCGCAAAATCGCCGAGC |
| | GGGTCACCTTCGGCGCGCCGACACTGCCGATCGTCTCCACCCTCACCGGCCGGCTCCTCACCG |
| | CCGAGGACCTGGCCGACCCGGACTACTGGGTCCGCCACGTCCGCCACGCCGTCCGCTTCGCC |
| | GACGCCGTCCGGACCCTCGCGGCTCAGGGCGTCACGGCGTTCCTGGAAGTCGGCCCCGGCGG |
| | CCTGACCCCGATGGTCCAGGAGACCCTGGCCGACACGGAGGTGACGGCCGTCCCCGCCTTGC |
| | GCGCCGACCGCCCCGAGCCCGAGGCCTTCACCACCGCCCTGGCCCAGCTCCACGTCCACGGC |
| | ACCGCCCTCGACTGGAACGCCGTCTTCATCGGCACCGGCGCCCGGCGGATCGACCTGCCCAC |
| | CTACGCCTTCCAGCGGGAGTCGTACTGGCTGGAGCCGGCCGCCCGCGAGCCGCGCGAGCAGT |
| | CGGCGGCGGACGGGTGGGAGTACCAGGCCACCTGGGCCCCGCTCCCGGAGGCCACGACCGC |
| | TACCGCGCAGGGCCGTTGGCTGCTCCTCGCGCCATCCGGCCCGAGCGCGCAGGCCGTCGCCG |
| | GCCCGCTGCGGGAGGCGCTGGCGGCCCGGGGCGCGGACGTCGACCTGCTGGAGGTCGACCA |
| | GGCCGACGACCGGCAGACGCTCGCCGCCCGCCTGCGCGAGGCCGCCGACGGTGAGGTGTCG |
| | CTGCGAACGGTCGTGTCGCTGCTGGCCCTGGGCGACCGTCAGGGCACCGGCGGGGCGCACAC |
| | CCCGCTGCCCGCCGCCGTGGCGCTGACCCTCACCCTGGTGCAGGCCCTCGGCGACGCGGACA |
| | TCGAGGCGCCGCTGTGGTGCGCCACCCGTGGCGCGGTGTCGGTGGACGGCTCCGACGGGCC |
| | GACCAGCCCCGCCCAGGCCCAGGTCTGGGGCCTGGGCCGGGTCGCCGCCCTGGAGCACTCC |
| | GGCCGCTGGGGTGGCCTGGTCGACCTGCCACCGGCCGCAGGGCAGGGCGAGGACCGGCCGG |
| | TCCTCGACGAGCGGGCGCTGACCCGGCTGGTCGCCGTCCTCACGACGGACGATGACGAGGAC |
| | CAGTTGGCCGTCCGCGACACCGCGGTCCTGGCCCGGAGGTTGACCCGCCTCGCCCCCGCCGA |
| | CCCCACCGCGCAGCCCGCCGACGGGGGTCCCGGCACCGACGGGTGGACGCCGCGCGGCACC |
| | GTCCTGGTCACCGGTGGCACCGGCGCGCTCGGCGGCCACGTCGCCCGCTGGCTGGCCGGCC |
| | GCGGCGCCGAGCACCTCGTGCTGACCAGCCGGCGCGGCCCCGCCGCCCCGGGCGCGGACGA |
| | GCTGCGCGACGAGCTGGAGCGGCTCGGCGCCCGCGTGACGGTCGCCGCCTGCGACGCCGCG |
| | GACCGCGACGCGCTGGCGCGGCTGCTGGCCGAACTCCCGGGCGAGCAGCCGCTCACCGCCGT |
| | GGTCCACACGGCCGGCGTCGACACCCCGGAGCTGCTCGCCGACGCCACCCCGGAGCGCTTCG |
| | CCGCCGTGGTGGCGGCGAAGGCCGCGGGCGCGTCCCACCTGGACGAGCTGCTGGGTGATCA |
| | GGACCTGGACGCCTTCGTGCTCTTCTCCTCGATCGCCGGGGTCTGGGGCAGCGGCGGCCAGG |
| | GCGCGTACTGCGCCGCCAACGCCCACCTGGACGCGCTCGCCGAGGCGCGCCGGGCCCGCGG |
| | CCTGGCGGCGACCGCCGTCGCCTGGGGCCCGTGGGCCGAGTCGGGCATGGCCGGCTCCGCC |
| | GAGGTCGCCGACGGCCTGCGCCGCCGGGGCCTGCGTCCGCTGGCACCGGACGCCGCGATCG |
| | CCGTACTGGCCGGGCTGCTGGTGCGCGGCGAGACCGCCGTGACGGTGGCCGACGTGGCGTG |
| | GGAACGGTTCGCGCCCGCGTTCACCGTGGTGCGGCCCAGCCCGCTGCTGTCGGACCTGCCCG |
| | AGGCCAGGGCGGCCCTGCGGCCGGCCGTGGACCCGCGCGACGCCGACCTCTCCGACGAGGC |
| | CGCCGAACTGCGCCGCACCCTCGCCGGGACCGACCCGGCCGAGCGGGAGCGCACCCTGGTG |
| | GAGCTGGTCCGCGCCGAGGCGGCGGCCGTGCTCGGGTTCCGCGGTGCCCACCTGGTCGACCC |
| | CGGGCGGCCGTTCCGGGAGCTGGGCTTCGACTCGTTGACGGCCGTCGAGCTGCGCAACCGGC |
| | TCAAGCGCGCCACCGGCGTCCGCCTGGCCACCACCGTGGTGTTCGACTACCCCACGGCCGTC |
| | GCGCTGGCCGGACAGCTCCTGACCGAGCTGCTGGGCGCGACGTCCGAGACCGGCGCCGGCG |
| | CGGGGACCGGCCGGGCCGCCCCGGACGAGCCGATCGCGATCGTGGCGATGGGCTGCCGCTT |
| | CCCCGGCGGCGTGCGCAGCCCCGAGGACCTGTGGCAGCTGGTGGCCGGGGAGACCGACGCG |
| | ATGTCGGACTTCCCCTCCAACCGGGGCTGGGACCTGGACGCGCTCTACCACCCCGACCCCGAC |
| | CACCCGGGCACCACCTACGCGCGCTCCGGCGGCTTCCTCCACGACCTGGCCGACTTCGACGCC |
| | GCCTTCTTCGGCATCTCGCCGCGCGAGGCCGTCGCCATGGACCCCCAGCAGCGGCTCCTCCTG |
| | GAGACCTCCTGGGAGGTGTTCGAGCGGGCGGGGATCGACCCGACGACGCTGCGCGGCACCC |
| | CGACCGGCGTCTTCGTCGGCACCGGCTACCAGGACTACCTCCAGCGCGGCCTGGACGTCCCG |
| | GACGGCGTGGTGGGCTACCTGGGCACCGGCAACGCGGCCAGCGTGGTGTCCGGGCGCATCTC |
| | CTACACCTTCGGTCTGGAGGGCCCGGCGGTCACCGTGGACACCGCCTGCTCCTCCTCCCTCGT |
| | CGCCCTGCACATGGCCGTCCAGTCGCTGCGCAACGGCGAGTGCACGCTGGCGCTGGCCGGCG |
| | GTGTCACCGTGATGTCCAACTCGGGCGCGTTCGTGGAGTTCAGCCGTCAGCGGGCGCTGTCC |
| | GCGGACGGCCGCTGCAAGGCGTTCTCCGCCGCCGCCGACGGCACCGGCTGGGGCGAGGGCG |
| | TCGGCCTGCTGCTGGTCGAGCGGCTCTCCGACGCCGAGCGCAACGGCCACCGCGTGCTGGCC |
| | GTGGTGCGCGGCACCGCGATCAACCAGGACGGCGCGTCCAACGGCCTCACCGCGCCCAACGG |
| | CCCCGCCCAGCAGCGGGTGATCCGCCAGGCCCTGGCCAACGCGGGCCTGACCGCGGACCAG |
| | GTCGACGCGGTGGAGGCGCACGGCACCGGCACCACGCTGGGCGACCCGATCGAGGCCCAGG |
| | CCCTGCTCGCCACCTACGGCCAGGAGCACCCCGAGGACCGTCCGCTGTGGCTGGGCTCGCTG |
| | AAGTCGAACATCGGCCACACCCAGGCCGCGGCCGGGGGGGGCGGCATCATCAAGATGGTCCA |
| | GGCCCTGCGGCACGGGCTGCTGCCGAAGACCCTGCACGTGGACGAGCCCACCCCGCACGTCG |
| | ACTGGTCGGCGGGCGCGGTGAAGCTGCTGACCCAGCCCGTGGCGTGGGCACCGGGCGACCG |
| | TCCCCGCCGGGCCGGCGTCTCCTCCTTCGGGATGAGCGGCACCAACGCCCACGTGATCATCGA |
| | GGAGCCCCCCACCGCGCGCCCGGCGGCCGACACCTCCGCCCCGGCGGCCGACGCCGCGCCG |
| | CGGCCGGTGCCGCTCGGCCCGGCGGCGGTGGCCCTGCCGCTGGCCGCCCGTACGGTCGGCG |
| | CGCTGGAGGAGCAGGCGGCCTCGCTCGCCGCACTGCTGCGCGCGCGGCCCGACCTGCGCCC |
| | CGAGGACGTCGGCTACTCGCTCGCCACCACGCGCTCGGCCTTCGAGCAGCGCGCGGTGGTGG |
| | TCGGCGCCGACCGGGCCGAGCTGACCGAAGGGCTGGAGACGCTGGCCGCCGGCGGCTCCGC |
| | CCGCCTGGTCGCGCGCGGCACCGCCGGAGCCGAACCGAAGGCCGTGTTCGTCTTCCCCGGCC |
| | AGGGCTCGCAGTGGGCCGGCATGGCCGTCGAACTGCTGGACACCTCCCCGGTGTTCCGGGAC |
| | GGCTTCGCCGCCTGCTCCCGGGCTCTGGCCCCGTTCGTGGACTGGAGCCCGGAGGACGTGCT |
| | GCGCGAGGCGCCCGGGGCGCCCGGGCTCGACCGGATCGACGTGATCCAGCCGGTGCTCTGG |
| | GCGGTCATGGTGTCGCTCGCCGAGCTGTGGCGCTCCTGCGGGGTGCGGCCCGCCGCCGTGGT |
| | GGGCCACTCGCAGGGCGAGATCGCCGCCGCCTGCTGCGCGGGCGCGCTGTCGCTGGAGGAC |
| | GGCGCGCGGATCGTGGCGCTGCGCAGCAGGCTGCTCTCCCGGCTGAGGGGCGGGATGGCCT |
| | CGATCACGCTGCCGCGGGCGGAGGTCTCCCGGCGCCTGGAGCGATGGGAGGAGCGCCTGAG |
| | CGTCGCCGCGGTCAACGGCCCGCTCAGCGTGGTGGTCGCCGGCGACGTCGACGCCCTGGACG |
| | AGCTGCTGGCCGCCTGCGAGGCCGAGGGGGTGGCGGCCAAGCGGATGGTCCGGGTCGAGTG |
| | CCCCTCGCACTGCCCCCAGGTCGAGGTGCTGCGCGACGAGCTCGACGCGATGCTCACCGGGA |
| | TCACCCCGAAGGCGCCGGAGGTGCCGTTCTACTCCACGGTGACCGGCGACAGGCTGGAGACC |
| | GCGCCGGACGCCGCCTACTGGTACGGAAACCTGCGCCGGACCGTGCAGTTCGAGCGGGCCAC |
| | CCGGCAGCTGCTCGCCGACGGCCACCACGTCTTCGTCGAGATCAGCCCGCATCCGGTGCTCGC |
| | CTTCGGCCTCCAGGAGACCATCGACGCCGTAGCGGGGGAGGAGCAGGAGGAGCAGGACGCC |
| | GCCCCGCAGCCGGCCCTGCCGCCGGTCGTGGTGCCCACCCTGCGGCGCGGCGACGGCGGCG |
| | CCCGGCGCTTCCTGGCCTCGCTGGGCGAGGCGCAGGCCAACGGCGTCCGGCCCGACTGGTCC |
| | GCGGTCTTCGCCGGTTGGGACGCCCGGGAGGTCGACCTGCCGACCTACCCGTTCCAGCGCCG |
| | GCGGTACTGGCTGGAGACGGCCGAGCGGCCCGCCGGGCCGGTGGCGGCCTCGGAGGTGGAC |
| | GCCGAATTCTGGGCGGCCGTCGAACGCGAGGACCTGACGGCGCTGACCGGGGACCTGCGCCT |
| | GGCGCAGGACGCCACGCTGCGCTCGGCCCTGCCCGCGCTCTCCTCCTGGCACCGGCGCCGCC |
| | GCGAGGAGTCCGTGGCGGACGGCTGGCGCTACCGGATCACCTGGCAGTCCGTCCCGGACCCC |
| | GCGGCGCCCGTCCTGGCCGGTACCTGGCTGGTGGTCCTGCCGCAGGAGCCGTCCGCCGACCC |
| | CGCCGTCGAACTGGCGCTGAACGCCCTGGCGACGCACGGAGCCGACGTCGTCCGGCTGACCG |
| | TGTCCGACGGCGAGGACCGGCAGGCGCTGGCCGGGCGGCTGGCCGAGGCCCTCGCCGGCCT |
| | CGGCGAGGTCGGCCCCGGCGGTGTGCTGTCGCTGCTCGCGCTCGACGAGCGGCCCCACCCCG |
| | AGCACCCGGGCCTGCCGCTGGGCCTCGGCCGGACCCTGACCCTGGTGCAGGCGCTCGGCGAC |
| | GCCGGAATCGCGGCCCCGCTGTGGTGCGCCACCCGGGCGGCGGTCTCCACCGGCGCCCAGG |
| | ACCCGCTGCGCGGCACCGCGCAGGCGCAGGTCTGGGCCCTGGGGGGGGTCGCCGCCCTGGA |
| | ACTGCCCGCCCGCTGGGGCGGCCTGGTCGACCTGCCCGACGGCGTGTCGGCCGCGGACGGC |
| | CGGATCGCCCGCCGCCTGGCCGCGGTGCTCTGCGGCGCCACGGCCGAGGACCAGGTCGCGG |
| | TGCGGGCCGGGGGCAGCTACGCCCGACGGATGGTGCGGGCCCCGCGCCCGGCCGGCGCCGC |
| | GCCGCGGCCCTGGCAGGTGCGCGGCACCGTGCTGGTCACCGGCGGCACCGGCTTCATCGGC |
| | GGCCAGGTGGCCCGCGCGCTCGCGGCCGACGGTGCCGAACACCTGGTGCTGGCCAGTCGCG |
| | CCGGCGCCGCCGCCGAGGGCGCCGAGCAGCTGCGCGGCGAACTGGAGGCGCTGGGCGCCAA |
| | GGCGACGATCACCGCGTGCGACCTGGCCGACCGGGCGGCGGTCGCCGAACTGGTGGCCACG |
| | CTGGACGACCTGGCGCCGCTGAGCGCCGTGGTGCACGCCGCCGGAGTGGGCGGCGCGCCCG |
| | CGATGCTCGCGGACACCACCGAGGCGGACGTCGCCGCCCTGCTGGGCGCGAAGGCGCTGGG |
| | CGCCGCGCACCTGGACGAACTGCTGGGCGAACGCCCGCTGGACGCCTTCGTGCTCTTCTCCTC |
| | CGGTGCCGCGGCCTGGGGCAGCGGCGCGCAGGCCGGCTACGCGGCCGCCAACGCCTTCGTC |
| | GACGCGCTGGCCGAGGACCGTCGCGCCCGCGGGCTGGCCGCCACCTCCGTCGCCTGGGGCG |
| | CCTGGGGCGGCGGCGGCATGGTCGACGCCGCTTCGGAGGAGCGCCTGCGCTCGCGCGGCCT |
| | GGACCCGATGAGCCCCGAGCTCGGGGTCCTGGCGCTGCGCCGCGCGGTGGAGTGCGAGGAC |
| | ACCACGCTGGTGGTCGCCGCCGTGGACTGGAAGCGCTTCGTGCCCGGCTTCACCGCGGCCCG |
| | GCCCAGCCCACTGATCGGCGACCTGCCCGAGGTCGCGCGCCTGCTCGCCGCCCAGGAGGCCC |
| | AGGACGACACCGAGGACGGTGCCGAGCCCCCGCTGCTGCGCCGGCTCGCCTCCCTCGCCGTC |
| | ACCGAACACGCCTCGGAGATCGTGGAGTTCGTCCGCGGCGAAGCCGCCGCCGTGCTCGGCCA |
| | CCCCTCGAAGGAGGCGGTCCAGGCGCAGCACGTCTTCCTGGAACTGGGCTTCGACTCGCTCAC |
| | CGCGGTCGAGTTCGGCAAGCGGCTCAGCCGGGCCAGCGGCCTGCGGCTGCCGAGCACACTG |
| | GCCTTCGACCACCGCACCCCGGTCGAGGTCGGACGGCGGCTGCAGTCCCTGCTGGCGGAGCG |
| | CGGCGGCCCCGGGGCCCCGGCCGCGGGAGCGGGCGCCGGCACGGGCTCGCCGGAGCCGTC |
| | GGAGTCGCAGGACCTCATCGTGTCGCTCTACCGCCGGGCCGGTGAACTCGGCCGGCTGACGG |
| | AGGGGATCGAACTGGTCACCGCCGCCGCCCGGTTGCGGCCGGTCTTCGACGCGGCGTCCGCC |
| | GCCGACCACCTGCCCGAGCCGGTGCAGTTCGCGCAGGGCCAGGACGGCCCGGCGCTGGTCTG |
| | CCTGGGCCCGTACATGGCGCCGTCGGGAGTCCACCAGTACGCCCGGTTCGCCGCCGCGTTCG |
| | GCGGCCGGCGCGGCATCTGGGCGCTGCCCGAGCCGGGCTTCGCCCCGGGGGAGGAGCTGCC |
| | CGGGGACGTCGAGGCGCTGATCGAGGCGCACGCCACGGCCATCGAGCGCAGCGTGGGCCAT |
| | CAGCCGGTCGTGCTGGTCGGCTACTCCTCGGGCGGCTGGGTCGCGCACGCGGTCGCCTGCCA |
| | CCTGGAGCGGATCGGCCGTCCGGTGGCGGGGATCGTCATGCTGGACAGCTTCACCCGGCGTC |
| | AGCGCATGGGCGAGCGGTTCCAGTCGGCGGTGGTGGAGGGGCAGTCGGAGCGCTTCGAGTT |
| | CGTCTCCGCGCCGGGCACCCAGCTGACCGCGATGGGCGGGTACCTGCGGATCTTCGAGGACT |
| | GGGACGCCCCGCGGGCCGCCGCCCCGACGCTGGTCGTCCGGGCCGCGGACTGGATGGCCAG |
| | GAACGAGACGGACGCGGACGACCGCCCCGCCGCCCCCGAGCACGCCGACACCGTGACCGAG |
| | GTGCCCGGCAACCACTACACCCTGATGGAGCGTCACTCCAACGAGGCCGCCGCGGCGGTCGA |
| | CGAGTGGCTGGACAGGCTCAGCCCGGTGGGGGAGTGA |
| 4 | AAACTCAAAGGAATTGACGGGGGCCCGCACAAGCAGCGGAGCATGTGGCTTAATTCGACGCAA |
| | CGCGAAGAACCTTACCAAGGCTTGACATATGCCGGAAAACCGTGGAGACACGGTCCCCCTTG |
| | TGGTCGGTATACAGGTGGTGCATGGTTGTCGTCAGCTCGTGTCGTGAGATGTTGGGTTAAGTC |
| | CCGCAACGAGCGCAACCCT |
| 5 | AAACT[C/T]AAA[T/G]GAATTGACGG |
| 6 | GTTGCTCGCGTTGGGA |

### EXAMPLES

The present invention will now be further exemplified with reference to the following examples. The present invention is in no way limited to the given examples or to the embodiments presented in the figures.

### Example 1: Growth Inhibition of the Plant Pathoqen Fusarium graminearum in Culture

The phytopathogenic fungus Fusarium graminearum is known worldwide to be the predominant causal agent of Fusarium head blight (FHB). F. graminearum parasitizes roots, stems, leaves, and reproductive tissues of many species of cereals and grasses, like wheat (Triticum aestivum), durum wheat (Triticum durum), barley (Hordeum vulgare) and oat (Avena sativa). Diseased spikelets exhibit symptoms of premature bleaching shortly after infection. As symptoms progress, the fungus colonizes the developing grain causing it to shrink and wrinkle inside the head. The fungus produces a mycotoxin known as deoxynivalenol that poses a significant threat to the health of domestic animals and humans. Management of the disease plays an important role in organic cereal production.

A co-culturing experiment of a bacterial strain member of the Kitasatospora-like subgroup with Deposit ID B/00227, B/00225, B/00228, B/00226, and B/00231 is executed with F. graminearum. Solid NA medium is prepared and dispensed over petri dishes with a diameter of 8 cm. Each purified bacterial strain is cultured in a liquid Luria broth culture until a dense bacterial culture is obtained. An amount, in particular 10 μl, of the liquid culture is taken and inoculated at 2.5 cm from the center of the petri dish. The petri dish is incubated at 28° C. overnight. Thereafter, the center of the petri dish is inoculated with 15 μl of a liquid culture incubated with F. graminearum and incubated at 21° C. for at least three days. The petri dish is scored with score A, B, C or no effect depending on the fungal growth, wherein score A is given when fungal growth is limited to 1 cm from the fungal plug, score B is given when fungal growth is observed up to 2 cm from the fungal plug, score C is given when the fungal pathogen reaches the bacterial growth line, and no effect is scored when the fungal pathogen outcompetes the bacterial growth (Table 2). Score A is given, as the edges of the F. graminearum growth is far from the bacterial growth line, more specific F. graminearum growth was only seen at less than 0.5 cm of the inoculated center.

TABLE 2

| | B/ | B/ | B/ | B/ | B/ |
|---|---|---|---|---|---|
| | 00227 | 00225 | 00228 | 00226 | 00231 |
| F. graminearum | A | A | A | A | A |

### Example 2: Co-Culturing Experiments with Funqal and Bacterial Pathoqens

The growth inhibition of bacterial strains with Deposit ID B/00227, B/00225, B/00228, B/00226 and B/00231 on different phytopathogenic fungi and bacteria was analyzed with a co-culturing experiment as described in Example 1, however, a different scoring system was used, scoring the inhibition of pathogenic growth around the assayed bacterial strain.

In this experiment, score A indicates a pathogenic growth inhibition zone around the bacterial strain of the invention of more than 15 mm (r>15 mm). Score B means that the growth inhibition zone around the bacterial strain was between 10 and 15 mm (15 mm>r>10 mm). Score C was given when r was 2 and 10 mm (10 mm>r>2 mm). Score D means that r was below 2 mm (r<2 mm). NA indicates that the combination has not yet been assayed.

TABLE 3

| | B/ | B/ | B/ | B/ | B/ |
|---|---|---|---|---|---|
| | 00227 | 00225 | 00228 | 00226 | 00231 |
| Ramularia collo-cygni | A | C | A | B | C |
| 1/10 NA | | | | | |
| Ramularia collo-cygni | A | C | A | NA | NA |
| PDA | | | | | |
| Sclerotinia sclerotiorum | B | B | A | A | A |
| 1/10 NA | | | | | |
| Sclerotinia sclerotiorum | C | C | B | NA | NA |
| PDA | | | | | |
| Fusarium oxysporum | B | C | B | B | D |
| 1/10 NA | | | | | |
| Fusarium oxysporum | C | C | B | NA | NA |
| PDA | | | | | |
| Fusarium | C | B | A | B | B |
| pseudograminearum | | | | | |
| 1/10 NA | | | | | |
| Fusarium | C | B | A | NA | NA |
| pseudograminearum | | | | | |
| PDA | | | | | |
| Rhizoctonia solani | D | B | D | B | C |
| 1/10 NA | | | | | |
| Rhizoctonia solani | D | C | D | NA | NA |
| PDA | | | | | |
| Botrytis cinerea 1/10 NA | B | B | B | B | B |
| Botrytis cinerea PDA | A | B | B | NA | NA |
| Colletotrichum | B | B | B | A | A |
| graminicola 10 NA | | | | | |
| Colletotrichum | B | B | A | NA | NA |
| graminicola PDA | | | | | |
| Microdochium nivale | B | B | B | A | A |
| 1/10 NA | | | | | |
| Microdochium nivale | B | C | B | NA | NA |
| PDA | | | | | |
| Gaeumannomyces | B | B | B | B | B |
| graminis var. | | | | | |
| tritici 1/10 NA | | | | | |
| Gaeumannomyces | B | C | B | NA | NA |
| graminis var. | | | | | |
| tritici PDA | | | | | |
| Tapesia yallundae PDA | B | B | B | A | A |
| Phytophthora infestans | NA | NA | B | A | A |
| PDA | | | | | |
| Rhizobium rhizogenes | A | B | B | A | A |
| 1/10 NA | | | | | |

These co-culturing experiments underline the broad-spectrum disease control in plants by the Kitasatospora strains of current invention. Said strains clearly inhibit the growth of the fungal and bacterial pathogens as listed in Table 3.

### Example 3: Biocontrol Expansion in Lactuca sativa

Lettuce plants from the nursery are planted in rows of 8×7 plants (1 experimental block). Replicates of 4 experimental blocks were assessed for the biocontrol strain B/00227. Biomass of the biocontrol strain was prepared at approximately 10\hat{ }8 CFU/ml. The biocontrol treatments were performed at 4 different time points: −1 (preventive), +3 (curative 1), +10 (curative 2), +17 (curative 3) days post planting. Rhizoctonia and Pythium diseases were naturally present in the soil of the greenhouse at the start of the experiment and artificially re-inoculated at 19 days post planting. As a positive control a chemical treatment (mix of Signum and Ortiva) was applied in accordance to the guidelines of the manufacturer.

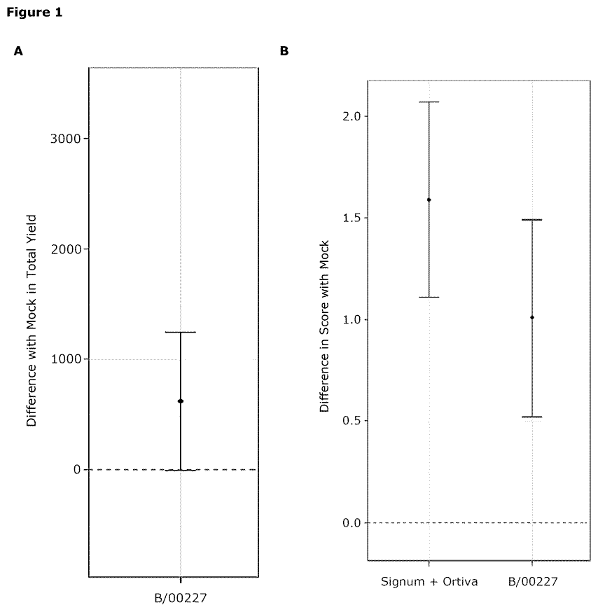

Marketable yield per experimental block was determined by weighing the fresh weight (in g) of the disease free part of the crops (i.e. diseased leaves were removed) treated versus the mock-treated crops (see dashed line) (FIG. 1A). Scoring of the total disease (Rhizoctonia+Pythium) was done at 30 days post planting by determining the level of infection and disease development in the treated versus the mock-treated plants (FIG. 1B). This assessment shows the biocontrol activities of B/00227 against the Rhizoctonia and Pythium pathogens, and substantiates its broad-spectrum pathogen control. Similar results were obtained for strains B/00225, B/00228, B/00226 and B/00231 (data not shown).

### Example 4: Reduction of Fusarium sp. in Triticum sp. by Application of Living Cells

Wheat cultivar Tybalt plants are grown till flowering stage in the greenhouse and transported to the growth chamber (temperature of 25° C. from 06h00 to 22h00 and 17° C. from 22h00 to 06h00 and relative humidity of 65-75%). Biomass of biocontrol strains are prepared at 10\hat{ }6-10\hat{ }8 CFU/ml. Biocontrol treatments were performed at 2 different time points: −2 (preventive) and +2 (curative) days post inoculation with Fusarium graminearum or Fusarium culmorum. The reference strains F. graminearum BRFM1977 or F. culmorum BRFM 1944 were used at 6×10\hat{ }5 CFU/ml and 2×10\hat{ }5 sp/mL respectively. As a positive control a chemical treatment (Prosaro) was applied in accordance to the guidelines of the manufacturer. The treatments were replicated on up to 50 different wheat spikes.

In addition to the reference strain (BRFM1977), two other F. graminearum strains, namely BRFM1982 and BRFM1995 which exhibit stronger aggressivity, were tested against the strains B/00227, B/00225, B/00228, B/00226 and B/00231. In all cases, the strains showed strong inhibition of disease.

Scoring of level of infection and disease development was done after 20 to 28 days post infection with the pathogen in the treated versus the mock-treated plants.

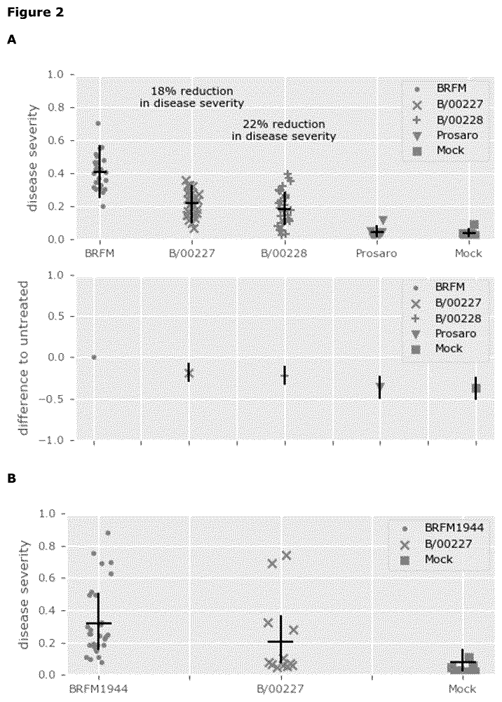

As visualized in FIG. 2A strains B/00227 and B/00228 inhibit the growth of F. graminearum after 20 dpi. The absolute inhibition of F. graminearum is approximately 18 to 22% compared to the non-treated T. aestivum spikes (see FIG. 2A) and more than 50% relatively (from 0,4 disease severity in the BRFM1977 treated only to 0, 18 disease severity in the B/00228 treated spikes). This inhibition was calculated on the assessment of 54 and 55 repeated spike treatments for B/00227 and B/00228 respectively. This assessment substantiates the biocontrol activity of B/00227 and B/00228. Moreover, the biocontrol activity of B/00227 was also demonstrated against Fusarium culmorum at 17 days post Fusarium culmorum infection (+17 dpi; FIG. 2B).

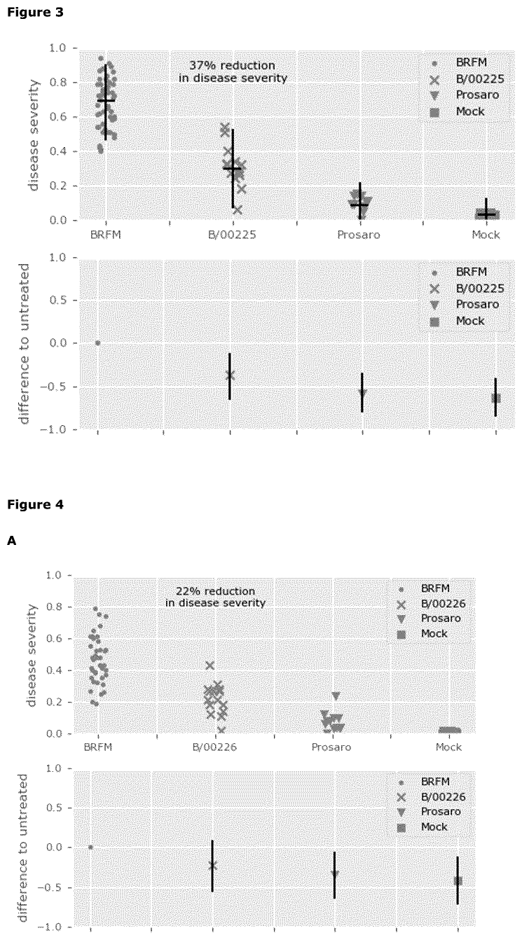

Similar results are seen in FIG. 3 for the purified bacterial strains deposited as B/00225, as biocontrol agent. After 21 dpi a clear inhibition of pathogen growth is established as the disease severity is about 40% less than the non-treated samples The disease inhibition of B/00225 comes close to the inhibitory activity observed with the chemical positive control Prosaro (lower graph, difference to untreated).

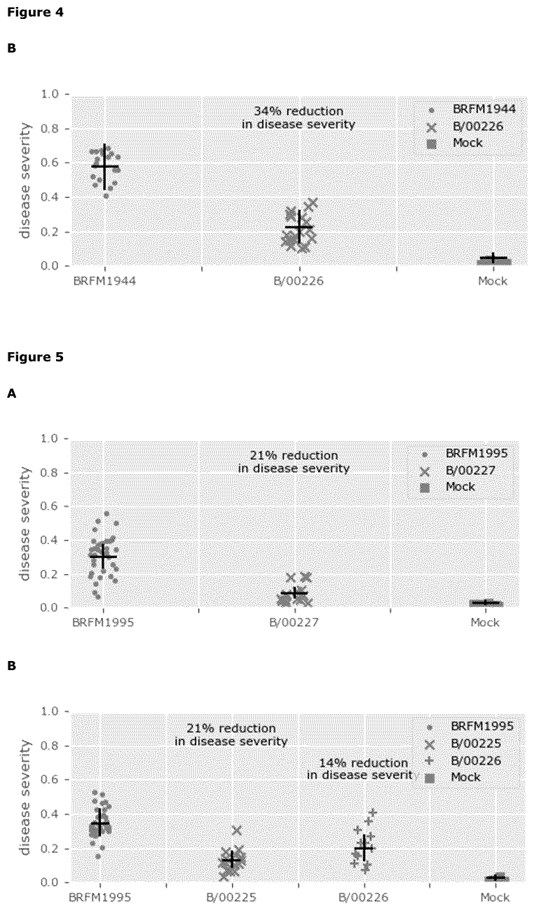

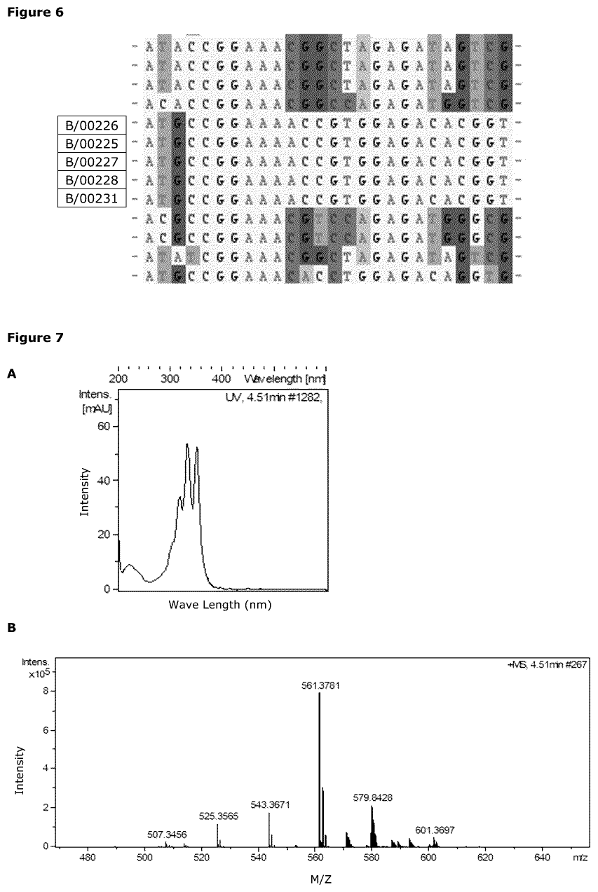

FIG. 4 visualizes the biocontrol activity of B/00226 against F. graminearum in 15 infected T. aestivum spikes after 22 dpi. The infected T. aestivum spikes show a disease severity of about 45%, whereas the B/00226 treated T. aestivum spikes only show a disease severity of about 20%. FIG. 6 also visualizes the biocontrol activity of B/00226 against F. culmorum in 18 infected T. aestivum spikes after 7 dpi. The infected T. aestivum spikes show a disease severity of about 58%, whereas the B/00226 treated T. aestivum spikes only show a disease severity of about 21%.

Also the biocontrol activity of B/00231 was tested and confirmed in a similar manner (data not shown).

The purified bacterial strains of current invention show both in vitro and in vivo broad-spectrum pathogen control resistance against plant pathogens. These biocontrol activities impart beneficial properties, as the plants treated with said strains show a reduced disease severity.

### Example 5: Reduction of Fusarium sp. in Triticum sp. by Application of Spores

FIG. 5 visualizes the biocontrol activity of B/00225, B/00226 and B/00227 when applied as spores against F. graminearum. FIG. 5A shows the infected T. aestivum spikes show a disease severity of about 34% when treated with Fusarium BRFM1995 only, whereas the B/00227 (on 16 spike replicates) treated T. aestivum spikes only show a disease severity of about 8% (after 19 dpi). FIG. 5B also visualizes the biocontrol activity of B/00225 (10 replicates) and B/00226 (12 replicates) against F. graminearum after 12 dpi. The infected T. aestivum spikes show a disease severity of about 35%, whereas the B/00225 and B/00226 treated T. aestivum spikes only show a disease severity of about 13% and 20% respectively.

Also the biocontrol activity of B/00228 and B/00231 was tested and confirmed in a similar manner (data not shown).

### Example 6: In Silico Identification of the Nucleotide Signature Sequence and Closely Related Strains with Broad Antifungal Activity

After identification of strain B/00227 and confirming the broad antifungal activity of the aforementioned strain, a whole genome sequencing was performed to define the genetic structure of the strain. Within the 16S sequence, a very conserved box, referred to as nucleotide signature sequence, was identified in almost middle portion of the 16S sequence. In silico studies further continued to explore the in house microbial collection database to identify very closely related strains. Four other strains namely B/00225, B/00228, B/00226 and B/00231 were identified in this way.

Further antifungal activity assessments proved that all four strains also show strong antifungal activity. Although no experiments are performed to demonstrate the direct functional involvement of the signature box in broad antifungal activity, it is clear that having a 16S nucleotide sequence bearing the highly conserved signature box delineates the broad antifungal activity of all five strains (FIG. 6).

### Example 7: In Silico Identification of the Type 1 Polyketide Synthase, a Gene Involved in the Production of Takanawaene

Streptomycetes produce a variety of secondary metabolites that can have antibacterial, antifungal or other specific antibiotic activity. The majority of these compounds fall within defined chemical categories which share a structural backbone and also share similarities at the gene and pathway level. These similarities can be exploited to annotate newly sequenced genomes and identify genes and gene clusters that potentially produce compounds within those known classes.

To discover a gene or genes responsible for antifungal activity of the bacterial strains B/00227, B/00225, B/00228, B/00226 and B/00231, DNA of the bacterial strains was extracted and sequenced using the Whole Genome Sequencing (WGS) method of Illumina. Next, the sequencing data was assembled into contigs and antiSMASH (https://antismash.secondarymetabolites.org/) was run to identify potential Biosynthetic Gene Clusters (BGC) that are associated with the production of known antibacterial secondary metabolites. In brief, antiSMASH works by detecting specific signatures (i.e. short amino acid stretches) that are specific to proteins involved in the production pathway of compounds in certain chemical classes e.g. polyketides, non-ribosomal proteins, terpenes.

Evidence from extract screening suggested that a polyene macrolide, Takanawaene, in particular Takanawaene C, is the causative agents for the observed antifungal activity of strains B/00227, B/00225, B/00226, B/00228 and B/00231. Polyene macrolides have known biochemistry and the large, defining central ring structure is produced by an enzyme known as a Type 1 Polyketide Synthase (T1PKS) or a complex thereof. Variation in the amino acid sequence of T1PKS translates in variation of the ring structure, which makes the T1PKS sequence uniquely linked to the chemical structure of the polyene macrolide. Type 1 PKS enzymes are well studied and their characteristic signatures are known, which allows for straightforward identification of their sequences in the genome. Thus, the T1PKS becomes a key or central gene for the target compound.

To identify a T1PKS, shared by strains B/00227, B/00225, B/00228, B/00226 and B/00231, the genes bearing the characteristic T1PKS signature were pooled and were aligned against each other (BLAST). T1PKS sequences that were shared between B/00227, B/00225, B/00228, B/00226 and B/00231 and had 100% sequence identity across the alignment were retained.

The Biosynthetic Gene Cluster (BGC) and T1PKS responsible for encoding Takanawaene C is not known, but the T1PKS of similar compounds are known. In relation to the T1PKS, similarity at the level of the central ring structure is a defining factor particularly the amount of double carbon bonds. By comparing the T1PKS of known pentaene macrolides to the T1PKS shared by the strains, we determined that the T1PKS encoded by SEQ ID No. 3 is involved in the production pathway of Takanawaene. The shared gene coding for this T1PKS showed a homology of ˜77% with the known T1PKS gene responsible for the production of another pentaene 28-membered-ring macrolide called filipin.

### Example 8: Detection of Active Compound Using Antifungal Activity Assessment in Bacterial Strain Extract Fractions

To assess the antifungal activity of the extracts of bacterial strains B/00227, B/00225, B/00228, B/00226 and B/00231 full microbial or fractionated extracts of strains were screened in vitro for their activity in vitro against Fusarium graminearum, Zymospectori tritici and Puccinia striiformis. Extracts or extract fractions that showed activity against the plant pathogens were further analyzed to identify the active metabolite(s). The aqueous phase of the strain extracts was found to be strongly active against Fusarium graminearum and Puccinia striiformis (complete growth inhibition against both pathogens, see Table 4). Examination of the aqueous fraction using a combination of UV and mass spectrometry revealed a compound with a pentaene macrolactone ring and a mass formula of C₃₃H₅₄O₈. The UV spectrum of the metabolite showing suppressive activity against phytopathogens undeniably indicated the presence of 5 conjugated double bonds (FIG. 7A).

The UV spectrum combined with the mass spectrometry profile of the compound (FIG. 7B) confirmed the active compound to be Takanawaene, in particular Takanawaene C. This compound was identified in strains B/00227, B/00225, B/00228, B/00226 and B/00231. Macrolides represent a large group of natural products that have diverse biological activities such as antibacterial, antifungal, antiparasitic, immunosuppressive etc, and are synthesized mainly by bacteria and some fungi. A common structural feature of all macrolides is the presence of a macrolactone ring assembled via repetitive condensation of small carboxylic acids by the polyketide synthase (PKS) enzymes. Full biological activity of macrolides usually depends on the chemical groups such as sugars, hydroxyl, methyl etc. that are added to the macrolactone ring later in the process of macrolide biosynthesis.

Polyene macrolides are distinguished from the rest of the macrolides by the large, 20- to 44-member macrolactone rings in combination with a series of 3 to 8 conjugated double bonds, and are classified accordingly as trienes, tetraenes, pentaenes, hexaenes, heptaenes, and octaenes. The number of double bonds in the chromophore region determines the UV absorption spectra of the compound, which are represented by three characteristic peaks.

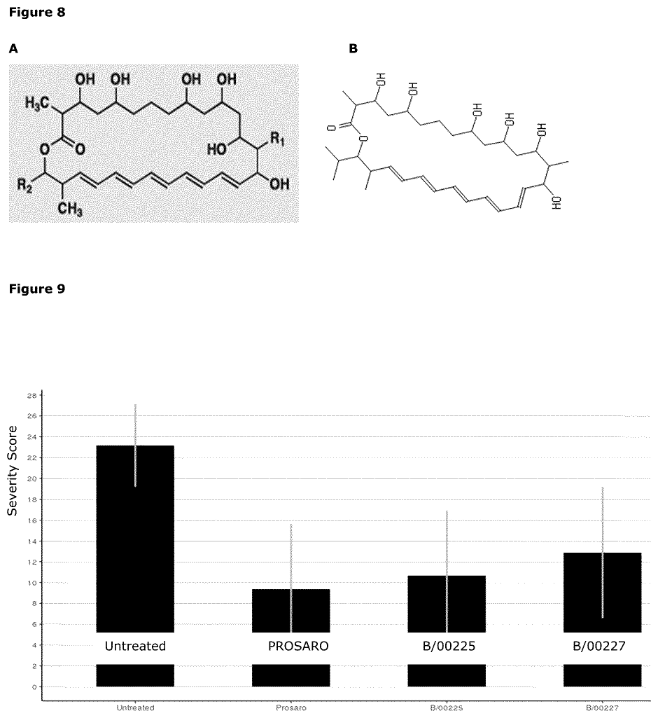

The chemical structure of Takanawaene (FIG. 8 A and B) containing a 28-membered ring macrolactone structure and 5 conjugated double bonds. The two functional groups R1 and R2 represent C—H moieties that can differ between different Takanawaenes. Takanawaene C typically contains —CH₃ and —CH(CH₃)2 respectively for R1 and R2. With this information, the dereplication was very robust to identify Takanawaene C as the active compound. This identification was very solid in terms of containing 5 conjugated double bonds (pentaene) as shown in the specific UV spectrum.

The combined results of strains extract antifungal activity and in silico analysis of WGS from example 7, strongly indicate that the compound responsible for antifungal activity of B/00227, B/00225, B/00228, B/00226 and B/00231 strains is Takanawaene C. This data discloses the antifungal activity of Takanawaene C as the active compound of bacterial strains B/00227, B/00225, B/00228, B/00226 and B/00231. There have been very few disclosures on antifungal activity of Takanawaene (A, B, or C), however, no disclosure of antifungal activity against phytopathogens in planta was ever reported. Moreover, the use of a microbial strain producing Takanawaene as active ingredient to control diseases in planta has never been reported (table 4).

### Example 9: Detection of a Novel Active Compound Using Antifungal Activity Assessment in Bacterial Strain Extract Fractions

To further assess the antifungal activity of the extracts of bacterial B/00227, B/00225, B/00228, B/00226 and B/00231 full microbial or fractionated extracts of strains were screened in vitro for their activity. Extracts or extract fractions that showed activity against the tested plant pathogens were further analyzed to identify the active metabolite(s). One of the fractions showed growth suppressive activity against Zymospetoria (63%) and Puccinia (12%) (Table 4).

In depth analysis of that fraction identified a compound for which the chemical formula was defined as C21H23NO4. A screening against the Natural Products Databases indicated that this is a novel, previously unknown, compound.

TABLE 4
Disease inhibitory activity as shown against Fusarium graminearum,
Zymoseptoria tritici and Puccinia striiformis in vitro
for Takanawaene C and the novel C21H23NO4 compound.

| | Fusarium | Zymospetoria | Puccinia |
|---|---|---|---|
| | graminearum | tritici | striiformis |
| Takanawaene | 100% growth | 9% growth | 100% inhibition |
| | inhibition | inhibition | |
| C21H23NO4 | 0% growth | 63% growth | 12% growth |
| | inhibition | inhibition | inhibition |

### Example 10: Reduction of the Funqal Disease Fusarium Head Blight Severity Caused by Fusarium sp. in Triticum sp. in Field Conditions

To evaluate the efficacy of the above-mentioned strains as biocontrol agents for Fusarium head blight (Fusarium graminearum) in wheat, Gedser wheat variety were tested in 24 m² plots in Beitem, Belgium. Treatments included two untreated, two mock treatments (formulation without microorganisms), one commercial chemical preventive fungicide treatment (PROSARO 250 EC) and 16 biocontrol treatments. Completely randomized factorial designs were used for 8 replicates of untreated treatment and four replicates for all other treatments.

Artificial infection of Fusarium graminearum: cereal grains were contaminated with inoculum of Fusarium graminearum. Infected grains were spread homogeneously all over the trial between the plant growth stage of TO (BBCH 30) and T1 (BBCH 32).

Biocontrol inoculation happened at two phases, first at BBCH 61 (10% anthesis), equivalent to the beginning of flowering and at BBCH65, equivalent to 50% anthesis. To assess phytotoxicity, a visual estimation was done 5-7 days after the first application of the treatments. Disease incidence was assessed (% plot affected by Fusarium) on a whole-plot basis by making an overall assessment of the mean percentage infection. Disease severity is defined by % of the spike affected by Fusarium, in 50-100 spikes/plot, as recommended at OEPP/EPPO (2012) guidelines. Both, disease incidence and severity were evaluated twice: at 2-3 weeks and 4-5 weeks after the first application of the treatments. All the assessments were performed in line with the OEPP/EPPO (2012) guidelines.

Untreated treatment showed a disease severity of 27%. The treatments B/00225 and B/00227 showed a disease severity of 10.6 and 13% respectively, which was significantly lower (P<0.05) than the untreated treatment. The commercial chemical fungicide PROSARO showed disease severity of 9.4% (FIG. 9).

Seed yield (ton/ha) was sampled from an area of 1.75 m×6 m in the central rows of each plot. Treatments B/00225 and B/00227 recorded 1 and 4% higher than the untreated respectively. PROSARO chemical control showed a seed yield 5.4% higher than Untreated (data not shown).

Similar results were retrieved for the other strains B/00228, B/00226 and B/00231 (data not shown).

The present invention is in no way limited to the embodiments described in the examples and/or shown in the figures. On the contrary, strains, populations, formulations, plant parts, compounds, uses and methods according to the present invention may be realized in many different ways without departing from the scope of the invention.

1. A purified bacterial strain for conferring a broad-spectrum pathogen control or resistance to a plant, wherein said strain comprises at least one 16S nucleotide sequence, said 16S nucleotide sequence shows at least 95% sequence identity to SEQ ID No 2 over the entire length of said sequence and comprises a nucleotide signature sequence according to SEQ ID No 1.

2. The purified bacterial strain according to claim 1, wherein said nucleotide signature sequence is located on the V6 region of said 16S nucleotide sequence.

3. The purified bacterial strain claim 1, wherein said 16S nucleotide sequence shows at least 99% sequence identity to SEQ ID No 2.

4. The purified bacterial strain according to claim 1, wherein said 16S nucleotide sequence is identical to SEQ ID No 2.

5. The purified bacterial strain according to claim 1, wherein said strain expresses a polyene having antifungal activity.

6. The purified bacterial strain according to claim 5, having in its genome a gene sequence having at least 75% of sequence identity with SEQ ID No 3, and wherein said gene sequence is involved in the production pathway of said polyene.

7. The purified bacterial strain according to claim 5, wherein said polyene is Takanawaene C.

8. The purified bacterial strain according to claim 1, wherein said strain produces a compound having the formula C21H23NO4.

9. The purified bacterial strain according to claim 1, wherein:
a. said purified bacterial strain is selected from Deposit ID B/00227, B/00225, B/00228, B/00226, or B/00231 obtainable from the Polish Collection of Microorganisms, under the terms of the Budapest Treaty, or
b. said purified bacterial strain is a mutant of a strain deposited under B/00227, B/00225, B/00228, B/00226, or B/00231, wherein said mutant is still capable of conferring a broad-spectrum pathogen control or resistance to a plant in a manner similar to said deposited strains, or
c. said purified bacterial strain has at least 98% genomic sequence identity with B/00227, B/00225, B/00228, B/00226, or B/00231, and is capable of conferring a broad-spectrum pathogen control or resistance to a plant in a manner similar to said deposited strains.

10. A bacterial population for conferring a broad-spectrum pathogen control or resistance in a plant comprising purified bacterial strains according to claim 1.

11. A formulation for conferring a broad-spectrum pathogen control or resistance to a plant, wherein said formulation comprises purified bacterial strains according to claim 1.

12. The formulation according to claim 11, wherein said purified bacterial strains are present at a CFU concentration of at least 10′CFU/ml when in a liquid formulation or 10′CFU/g when in a non-liquid formulation.

13. The formulation according to claim 11, wherein said bacterial strains are present as living cells or spores; and/or in a spray-dried or freeze-dried form.

14. A plant part, such as a shoot, root or seed, comprising the formulation according to claim 11.

15. A method for controlling a pathogen in a plant, comprising applying the purified bacterial strain according to claim 1 to said plant.

16. A method for conferring a broad-spectrum pathogen control or resistance in a plant, comprising artificially introducing a bacterial population or a formulation comprising bacterial strains according to claim 1 to a plant, a plant part or a substrate comprising or hosting said plant or plant part, thereby conferring a broad-spectrum pathogen control or resistance to a plant.

17. A method for controlling a pathogen in a plant, comprising applying a Takanawaene and/or a compound having the formula C21H23NO4 to the plant.

18. A method comprising deriving or obtaining a Takanawaene and/or a compound having the formula C21H23NO4 from the purified bacterial strain according to claim 1.

19. A compound having the formula C21H23NO4.

20. The method according to claim 17, wherein the pathogen is a microbe or fungus.

21. The method according to claim 17, wherein the plant is a crop.

22. (canceled)

23. The method according to claim 15, wherein said pathogen is selected from Fusarium spp., Phytophthora spp., Pythium spp., Rhizoctonia spp., Puccinia spp., Mycosphaerella spp., Sclerotinia spp., Botrytis spp., Rhizobium spp., Colletotrichum spp., Microdochium spp., Gaeumannomyces spp., Tapesia spp., Ustilago spp., Ramularia spp., or Zymoseptoria spp.


SEQUENCE LISTING (6 sequences)

SEQ ID NO: 1 - DNA, 16 bp, Unknown
  Bacteria
accgtggagacacggt

SEQ ID NO: 2 - DNA, 1522 bp, Unknown
  Bacteria
cacggagagtttgatcctggctcaggacgaacgctggcggcgtgcttaacacatgcaagtcgaacggtgaagcccttcggggtggatcagtggcgaacgggtgagtaacacgtgggcaatctgccctgcactctgggacaagccctggaaacggggtctaataccggatatgaccttcctccgcatgggggttggtggaaagctccggcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgcaagtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccccgggtctgcattcgatacgggcaggctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatatgccggaaaaccgtggagacacggtcccccttgtggtcggtatacaggtggtgcatggttgtcgtcagctcgtgtcgtgagatgttgggttaagtcccgcaacgagcgcaacccttgttctgtgttgccagcatgcctttcggggtgatggggactcacaggagactgccggggtcaactcggaggaaggtggggacgacgtcaaatcatcatgccccttatgtcttgggctgcacacgtgctacaatggtcggtacaaagggctgcgatgccgtgaggcggagcgaatcccaaaaagccggcctcagttcggattggggtctgcaactcgaccccatgaagttggagttgctagtaatcgcagatcagcatgctgcggtgaatacgttcccgggccttgtacacaccgcccgtcacgtcacgaaagtcggtaacacccgaagccggtggcctaacccgtaaggggaggagccgtcgaaggtgggaccagcgattgggacgaagtcgtaacaaggtagccgtaccggaaggtgcggctggatcacctccttt

SEQ ID NO: 3 - DNA, 10203 bp, Unknown
  Bacteria
atgcagaatgagcagaacgagcagaagctgctcgactacctcaagcgtgtgacggccgatctcaagcagacccggcgccggttgcacgaggtggagaccaaggaccaggagccgatcgccatcgtggcgatgggctgccgttaccccgggcaggtcaacagccccgaggagctctggcaactggtcgagcgcggtggtgacgcggtctccggcttccccaccaaccgcggttgggacctggaggcgctctacgacccggaccccgagagccccgggaagtgctacacccgcgagggcgggttcctgcacggggccggggagttcgacccgtcgttcttcgggatctcgccgcgcgaggcgctggccaccgacccgcagcagcggctgctgctggaggtcgcctgggagaccttcgagcgcgccggcatcgacccctcctcggtcaggggctcgaacaccggcgtcttcgtcggcctcgcctaccaggggtacgcgggcgacgaggagagcgcacaggagctggagggcttcctgctgaccggcaccgcgccgagcgtcgcctcgggccggctctcctacaccttcgggctcgaaggcccggcgctgaccgtggacaccgcgtgctcgtcctcgctggtcgccctgcacctggcgatccaggccctgcgccggcgggagtgcaccctcgcgctggccggcggtgcggcgatcatggcgagcaccggcatgttcaccgagttcagccgccagcagggcctcgccgtggacggccgctgcaagtccttcgccgccgaggcggacggcacgggctggggcgagggcgtcggcatgctgctggtcgagcggctctccgatgccgagcgcaacggtcacccggtgctggcggtggtgcggggttcggccgtgaaccaggacggcgcgtccaacggtctgacggccccgaacggccccgcccagcagcgggtgatccgtcaggcgttggccagtgcggggctgtcggcggcggacgtggacgtggtcgaggcgcacggtacggggacgacgctgggtgacccgatcgaggcccaggcgctgctggcgacgtacgggcaggaccggcccgaggaccgtccgctgtggctgggctcgttgaagtccaacatcggtcacacccaggcggcggccggggtgggcggcatcatcaagatggtgcaggcgatgcggcacggtgttctgccgcgcaccctgcacgcggagcagccgacgccgcacgtcgactggtcggcgggcgcggtacggctgctgaccgagccggtcgcctggccggagggcgaccgcccgcggcgggccgccgtctcctccttcggggtcagcggcaccaacgcgcacaccatcatcgagcaggccccggccgcccccgaggcggacgaggagccgcccgcccgggccgagccgacgcccgccgcggcgccgctgtcctgggtggtctccgcccgcagcagggccgccctgcgcggacaggcggaacggctgctctcccaggtgatcggcctgcccgacgcgcggccgctcgacgtcgcctactcgctggcgacctcccgtgccgccctggaccaccgcgccgcggtggtcggcacggacctcgccgagctcatcgacggcgtccgcgcgctggccgaggggaaggcggccccgcaggtgatccagggacacaccggccgggacgccgggctcgccctcgtgttctcggggcaggggagccagcggctggggatggggcgtgaactgtacgcgtccttcccggtgttcgccgaggcgttcgacgcggtgtgtgcggcggtggacgcccacctggaacgcccgctgcgcgaggtggtgttcggcgaggacgcggagctgctgaaccggacggagttcacccagccggcgttgttcgcggtggaggtggcgctcttccggctcaccgagtcgtggggggtgcgggcggacttcctggccgggcactcgatcggtgagttcgccgccgcccatgtggcgggtgttctgtcgctggccgatgcggccgaactggtcgtggcccgtggaaggttgatgcaggagctgccctcgggcggtgcgatggtggcggtgcaggcctccgaggaggaggtggcgccgctgctcgccggccgcgaggagcgcgtgggtatcgcggcggtcaacggcccgtcctcggtggtcatttcgggtgccgaggacgcggtggcggaggtggtggaggcgctggcggcggacggatgccggacgaagcgcctcgcggtctctcacgcgttccactcgccgctgatggagccgatgctcgcggccttccgcaaaatcgccgagcgggtcaccttcggcgcgccgacactgccgatcgtctccaccctcaccggccggctcctcaccgccgaggacctggccgacccggactactgggtccgccacgtccgccacgccgtccgcttcgccgacgccgtccggaccctcgcggctcagggcgtcacggcgttcctggaagtcggccccggcggcctgaccccgatggtccaggagaccctggccgacacggaggtgacggccgtccccgccttgcgcgccgaccgccccgagcccgaggccttcaccaccgccctggcccagctccacgtccacggcaccgccctcgactggaacgccgtcttcatcggcaccggcgcccggcggatcgacctgcccacctacgccttccagcgggagtcgtactggctggagccggccgcccgcgagccgcgcgagcagtcggcggcggacgggtgggagtaccaggccacctgggccccgctcccggaggccacgaccgctaccgcgcagggccgttggctgctcctcgcgccatccggcccgagcgcgcaggccgtcgccggcccgctgcgggaggcgctggcggcccggggcgcggacgtcgacctgctggaggtcgaccaggccgacgaccggcagacgctcgccgcccgcctgcgcgaggccgccgacggtgaggtgtcgctgcgaacggtcgtgtcgctgctggccctgggcgaccgtcagggcaccggcggggcgcacaccccgctgcccgccgccgtggcgctgaccctcaccctggtgcaggccctcggcgacgcggacatcgaggcgccgctgtggtgcgccacccgtggcgcggtgtcggtggacggctccgacgggccgaccagccccgcccaggcccaggtctggggcctgggccgggtcgccgccctggagcactccggccgctggggtggcctggtcgacctgccaccggccgcagggcagggcgaggaccggccggtcctcgacgagcgggcgctgacccggctggtcgccgtcctcacgacggacgatgacgaggaccagttggccgtccgcgacaccgcggtcctggcccggaggttgacccgcctcgcccccgccgaccccaccgcgcagcccgccgacgggggtcccggcaccgacgggtggacgccgcgcggcaccgtcctggtcaccggtggcaccggcgcgctcggcggccacgtcgcccgctggctggccggccgcggcgccgagcacctcgtgctgaccagccggcgcggccccgccgccccgggcgcggacgagctgcgcgacgagctggagcggctcggcgcccgcgtgacggtcgccgcctgcgacgccgcggaccgcgacgcgctggcgcggctgctggccgaactcccgggcgagcagccgctcaccgccgtggtccacacggccggcgtcgacaccccggagctgctcgccgacgccaccccggagcgcttcgccgccgtggtggcggcgaaggccgcgggcgcgtcccacctggacgagctgctgggtgatcaggacctggacgccttcgtgctcttctcctcgatcgccggggtctggggcagcggcggccagggcgcgtactgcgccgccaacgcccacctggacgcgctcgccgaggcgcgccgggcccgcggcctggcggcgaccgccgtcgcctggggcccgtgggccgagtcgggcatggccggctccgccgaggtcgccgacggcctgcgccgccggggcctgcgtccgctggcaccggacgccgcgatcgccgtactggccgggctgctggtgcgcggcgagaccgccgtgacggtggccgacgtggcgtgggaacggttcgcgcccgcgttcaccgtggtgcggcccagcccgctgctgtcggacctgcccgaggccagggcggccctgcggccggccgtggacccgcgcgacgccgacctctccgacgaggccgccgaactgcgccgcaccctcgccgggaccgacccggccgagcgggagcgcaccctggtggagctggtccgcgccgaggcggcggccgtgctcgggttccgcggtgcccacctggtcgaccccgggcggccgttccgggagctgggcttcgactcgttgacggccgtcgagctgcgcaaccggctcaagcgcgccaccggcgtccgcctggccaccaccgtggtgttcgactaccccacggccgtcgcgctggccggacagctcctgaccgagctgctgggcgcgacgtccgagaccggcgccggcgcggggaccggccgggccgccccggacgagccgatcgcgatcgtggcgatgggctgccgcttccccggcggcgtgcgcagccccgaggacctgtggcagctggtggccggggagaccgacgcgatgtcggacttcccctccaaccggggctgggacctggacgcgctctaccaccccgaccccgaccacccgggcaccacctacgcgcgctccggcggcttcctccacgacctggccgacttcgacgccgccttcttcggcatctcgccgcgcgaggccgtcgccatggacccccagcagcggctcctcctggagacctcctgggaggtgttcgagcgggcggggatcgacccgacgacgctgcgcggcaccccgaccggcgtcttcgtcggcaccggctaccaggactacctccagcgcggcctggacgtcccggacggcgtggtgggctacctgggcaccggcaacgcggccagcgtggtgtccgggcgcatctcctacaccttcggtctggagggcccggcggtcaccgtggacaccgcctgctcctcctccctcgtcgccctgcacatggccgtccagtcgctgcgcaacggcgagtgcacgctggcgctggccggcggtgtcaccgtgatgtccaactcgggcgcgttcgtggagttcagccgtcagcgggcgctgtccgcggacggccgctgcaaggcgttctccgccgccgccgacggcaccggctggggcgagggcgtcggcctgctgctggtcgagcggctctccgacgccgagcgcaacggccaccgcgtgctggccgtggtgcgcggcaccgcgatcaaccaggacggcgcgtccaacggcctcaccgcgcccaacggccccgcccagcagcgggtgatccgccaggccctggccaacgcgggcctgaccgcggaccaggtcgacgcggtggaggcgcacggcaccggcaccacgctgggcgacccgatcgaggcccaggccctgctcgccacctacggccaggagcaccccgaggaccgtccgctgtggctgggctcgctgaagtcgaacatcggccacacccaggccgcggccggggcgggcggcatcatcaagatggtccaggccctgcggcacgggctgctgccgaagaccctgcacgtggacgagcccaccccgcacgtcgactggtcggcgggcgcggtgaagctgctgacccagcccgtggcgtgggcaccgggcgaccgtccccgccgggccggcgtctcctccttcgggatgagcggcaccaacgcccacgtgatcatcgaggagccccccaccgcgcgcccggcggccgacacctccgccccggcggccgacgccgcgccgcggccggtgccgctcggcccggcggcggtggccctgccgctggccgcccgtacggtcggcgcgctggaggagcaggcggcctcgctcgccgcactgctgcgcgcgcggcccgacctgcgccccgaggacgtcggctactcgctcgccaccacgcgctcggccttcgagcagcgcgcggtggtggtcggcgccgaccgggccgagctgaccgaagggctggagacgctggccgccggcggctccgcccgcctggtcgcgcgcggcaccgccggagccgaaccgaaggccgtgttcgtcttccccggccagggctcgcagtgggccggcatggccgtcgaactgctggacacctccccggtgttccgggacggcttcgccgcctgctcccgggctctggccccgttcgtggactggagcccggaggacgtgctgcgcgaggcgcccggggcgcccgggctcgaccggatcgacgtgatccagccggtgctctgggcggtcatggtgtcgctcgccgagctgtggcgctcctgcggggtgcggcccgccgccgtggtgggccactcgcagggcgagatcgccgccgcctgctgcgcgggcgcgctgtcgctggaggacggcgcgcggatcgtggcgctgcgcagcaggctgctctcccggctgaggggcgggatggcctcgatcacgctgccgcgggcggaggtctcccggcgcctggagcgatgggaggagcgcctgagcgtcgccgcggtcaacggcccgctcagcgtggtggtcgccggcgacgtcgacgccctggacgagctgctggccgcctgcgaggccgagggggtggcggccaagcggatggtccgggtcgagtgcccctcgcactgcccccaggtcgaggtgctgcgcgacgagctcgacgcgatgctcaccgggatcaccccgaaggcgccggaggtgccgttctactccacggtgaccggcgacaggctggagaccgcgccggacgccgcctactggtacggaaacctgcgccggaccgtgcagttcgagcgggccacccggcagctgctcgccgacggccaccacgtcttcgtcgagatcagcccgcatccggtgctcgccttcggcctccaggagaccatcgacgccgtagcgggggaggagcaggaggagcaggacgccgccccgcagccggccctgccgccggtcgtggtgcccaccctgcggcgcggcgacggcggcgcccggcgcttcctggcctcgctgggcgaggcgcaggccaacggcgtccggcccgactggtccgcggtcttcgccggttgggacgcccgggaggtcgacctgccgacctacccgttccagcgccggcggtactggctggagacggccgagcggcccgccgggccggtggcggcctcggaggtggacgccgaattctgggcggccgtcgaacgcgaggacctgacggcgctgaccggggacctgcgcctggcgcaggacgccacgctgcgctcggccctgcccgcgctctcctcctggcaccggcgccgccgcgaggagtccgtggcggacggctggcgctaccggatcacctggcagtccgtcccggaccccgcggcgcccgtcctggccggtacctggctggtggtcctgccgcaggagccgtccgccgaccccgccgtcgaactggcgctgaacgccctggcgacgcacggagccgacgtcgtccggctgaccgtgtccgacggcgaggaccggcaggcgctggccgggcggctggccgaggccctcgccggcctcggcgaggtcggccccggcggtgtgctgtcgctgctcgcgctcgacgagcggccccaccccgagcacccgggcctgccgctgggcctcggccggaccctgaccctggtgcaggcgctcggcgacgccggaatcgcggccccgctgtggtgcgccacccgggcggcggtctccaccggcgcccaggacccgctgcgcggcaccgcgcaggcgcaggtctgggccctggggcgggtcgccgccctggaactgcccgcccgctggggcggcctggtcgacctgcccgacggcgtgtcggccgcggacggccggatcgcccgccgcctggccgcggtgctctgcggcgccacggccgaggaccaggtcgcggtgcgggccgggggcagctacgcccgacggatggtgcgggccccgcgcccggccggcgccgcgccgcggccctggcaggtgcgcggcaccgtgctggtcaccggcggcaccggcttcatcggcggccaggtggcccgcgcgctcgcggccgacggtgccgaacacctggtgctggccagtcgcgccggcgccgccgccgagggcgccgagcagctgcgcggcgaactggaggcgctgggcgccaaggcgacgatcaccgcgtgcgacctggccgaccgggcggcggtcgccgaactggtggccacgctggacgacctggcgccgctgagcgccgtggtgcacgccgccggagtgggcggcgcgcccgcgatgctcgcggacaccaccgaggcggacgtcgccgccctgctgggcgcgaaggcgctgggcgccgcgcacctggacgaactgctgggcgaacgcccgctggacgccttcgtgctcttctcctccggtgccgcggcctggggcagcggcgcgcaggccggctacgcggccgccaacgccttcgtcgacgcgctggccgaggaccgtcgcgcccgcgggctggccgccacctccgtcgcctggggcgcctggggcggcggcggcatggtcgacgccgcttcggaggagcgcctgcgctcgcgcggcctggacccgatgagccccgagctcggggtcctggcgctgcgccgcgcggtggagtgcgaggacaccacgctggtggtcgccgccgtggactggaagcgcttcgtgcccggcttcaccgcggcccggcccagcccactgatcggcgacctgcccgaggtcgcgcgcctgctcgccgcccaggaggcccaggacgacaccgaggacggtgccgagcccccgctgctgcgccggctcgcctccctcgccgtcaccgaacacgcctcggagatcgtggagttcgtccgcggcgaagccgccgccgtgctcggccacccctcgaaggaggcggtccaggcgcagcacgtcttcctggaactgggcttcgactcgctcaccgcggtcgagttcggcaagcggctcagccgggccagcggcctgcggctgccgagcacactggccttcgaccaccgcaccccggtcgaggtcggacggcggctgcagtccctgctggcggagcgcggcggccccggggccccggccgcgggagcgggcgccggcacgggctcgccggagccgtcggagtcgcaggacctcatcgtgtcgctctaccgccgggccggtgaactcggccggctgacggaggggatcgaactggtcaccgccgccgcccggttgcggccggtcttcgacgcggcgtccgccgccgaccacctgcccgagccggtgcagttcgcgcagggccaggacggcccggcgctggtctgcctgggcccgtacatggcgccgtcgggagtccaccagtacgcccggttcgccgccgcgttcggcggccggcgcggcatctgggcgctgcccgagccgggcttcgccccgggggaggagctgcccggggacgtcgaggcgctgatcgaggcgcacgccacggccatcgagcgcagcgtgggccatcagccggtcgtgctggtcggctactcctcgggcggctgggtcgcgcacgcggtcgcctgccacctggagcggatcggccgtccggtggcggggatcgtcatgctggacagcttcacccggcgtcagcgcatgggcgagcggttccagtcggcggtggtggaggggcagtcggagcgcttcgagttcgtctccgcgccgggcacccagctgaccgcgatgggcgggtacctgcggatcttcgaggactgggacgccccgcgggccgccgccccgacgctggtcgtccgggccgcggactggatggccaggaacgagacggacgcggacgaccgccccgccgcccccgagcacgccgacaccgtgaccgaggtgcccggcaaccactacaccctgatggagcgtcactccaacgaggccgccgcggcggtcgacgagtggctggacaggctcagcccggtgggggagtga

SEQ ID NO: 4 - DNA, 207 bp, Unknown
  Bacteria
aaactcaaaggaattgacgggggcccgcacaagcagcggagcatgtggcttaattcgacgcaacgcgaagaaccttaccaaggcttgacatatgccggaaaaccgtggagacacggtcccccttgtggtcggtatacaggtggtgcatggttgtcgtcagctcgtgtcgtgagatgttgggttaagtcccgcaacgagcgcaaccct

SEQ ID NO: 5 - DNA, 20 bp, artificial sequence
  primer sequence
  variation (6)..(6): C to T
  variation (10)..(10): T to G
aaactcaaatgaattgacgg

SEQ ID NO: 6 - DNA, 16 bp, artificial sequence
  primer sequence
gttgctcgcgttggga